(19) United States
(12) Patent Application Publication
(10) Pub. No.: US 2006/0286548 A1
(43) Pub. Date: Dec. 21, 2006

(54) Method of making recombinant human antibodies for use in biosensor technology

(76) Inventors: Gregory Liposky, Northborough, MA (US); Byron V. Olsen, Hopkinton, MA (US)

Correspondence Address: GTC BIOTHERAPEUTICS, INC,;C/O WOLF, GREENFIELD & SACKS, P.C., FEDERAL RESERVE PLAZA, 600 ATLANTIC AVE., BOSTON, MA, 02210-2206, US

(21) Appl. No.: 11/156,831

(22) Filed: Jun. 16, 2005

(51) Int. Cl. C12Q 1/70; G01N 33/554; C12M 1/34; G01N 33/543; G01N 33/569

(52) U.S. Cl. 435/5; 435/7.32; 436/518; 435/287.2

(57) ABSTRACT

According to the current invention and apparatus and methods are provided for the production of modified antibodies and calins for use in biosensor applications.

### FIELD OF THE INVENTION

The present invention relates to the production and use of recombinant human antibodies to detect the presence of certain molecules of interest including those that may case specific disease related pathologies. In particular the current invention provides for the production of human antibodies of interest for use in various biosensor detection apparati.

### BACKGROUND OF THE INVENTION

As stated above, the present invention relates generally to the field of the production of antibodies and the methods of using these antibodies for biosensor applications. Preferably those antibodies are produced from the milk of transgenic animals. More particularly, it concerns improved methods for generating large quantities of transgenic antibodies capable of detecting certain pathogenic agents of interest and thereafter reporting such an interaction.

Economics of Production

In vivo, antibodies are generated in animal cells in response to an antigen exposure and they contribute to the overall immune response. The use of these molecules as therapeutic agents, laboratory tools and in immunocytochemistry is very widespread and growing. One of the most significant problems with using antibodies is their cost. Owing to government and industry policies regarding animal rights, processing requirements and the expense of maintaining production facilities for antibody production in cell culture, the costs of production has greatly increased over the last several years. Moreover, the driving force for the development of large-scale production capabilities has been an understanding of the need for increasing the kind and quality of antibody therapeutic applications. Commercially the pharmaceutical industry can command higher prices per unit of product so the use of antibodies for non-therapeutic applications has also incurred substantial price increases as production resources have been turned towards more lucrative pharmaceutical efforts and capacity is used up.

Difficulties with Large-Scale Antibody Production in Prokaryotes

Bacterial systems that have proven to be very efficient for expression of some proteins have sometimes given disappointing results with recombinant antibodies. Efficiencies of recombinant antibody expression in E. coli vary with the antibody. In some early experiments expression of soluble Fabs and ScFvs was under 10 mg/l, which is considerably less than the amounts expected from flask cultures of hybridomas. This is sufficient for applications such as enzyme immunoassay and western blotting, but not for large scale pharmaceutical or industrial needs. Two known problems are misfolding of the antibody chains and incompatibility with bacterial secretory pathways.

Currently there are approximately six different antibodies approved by regulatory authorities for use as drugs against diseases such as cancer. It is thought that many more will be approved for medical purposes in the next decade, straining capacity and again pushing production prices. The current cost of antibody production using fermentation in animal cell cultures, is about US $333.00 /g. According to a preferred embodiment of the current invention the cost of antibody production in the milk of transgenic animals is substantially below US $100.00/g with an ease of upwards scaleability not seen by other production methods. Thus, transgenic production offers a more efficient production platform that can make widely available the unique properties and volumes of antibodies needed for use in biosensor and other applications at costs that are commercially more feasible.

Detecting Biological Pathogens

Biological pathogens (for example derived from anthrax bacteria or the hanta virus, respectively) contain toxins produced by biological organisms (for example, botulinus toxin) that can be released intentionally or accidentally. These same biotoxins, which can occur naturally, can result in disease, fear, disruption to society, significant economic harm and potentially large-scale loss of life. Individuals can be exposed to agents of this sort from inhalation, skin contact, or by the ingestion of contaminated food or water. After exposure to a pathogen or toxin, physical symptoms can be delayed and prove difficult to distinguish from naturally occurring illnesses as well as shielding the location or time of exposure. Similarly, crops or livestock can be exposed to pathological biologic agents in several ways rendering food production vulnerable to pathogenic organisms and again causing substantial societal disruption.

The potential reality of two kinds of biological terrorist events underlie the need for the current invention. The first is the release of communicable infectious agents—like smallpox, Ebola, or hanta virus—that can spread rapidly within communities through incidental contact and retain lethal potential. The second kind of threat consists of biological agents that may cause disease or death in individuals but generally are not transmitted between individuals-the most familiar example being anthrax. Though the greatest threats from these agents are when then they are airborne, it is also possible that they could arise from animal vectors or occur/persist in water supplies.

In either case, some agents may persist in the environment, as do anthrax spores, and continue to cause problems long after their release. In addition to these naturally occurring pathogens, weaponized biological agents can be genetically engineered to resist current therapies and evade vaccine-induced immunities. Preparedness for a biological attack against population centers, crops, or critical facilities is complicated by the large number of potential agents, the long incubation periods of some agents, and their potential for secondary transmission.

Moreover, these events raise serious concerns about how many outbreaks could be managed at one time. The terrorist attacks of Sep. 11, 2001, and the intentional release of anthrax spores shortly afterward in the USA also revealed significant vulnerabilities in public health and agricultural infrastructures. Technologies available to identify biological agents in air, soil, and water samples have had only limited success. Current invention air or water samples are collected and quickly identified, allowing a population to be warned of the pathogen's presence and allowing steps to be taken by the appropriate authorities to counteract any potential loss of life, exposure to the agent and/or general outbreak of a specific disease. However, existing technologies for rapid and reliable detection of pathogenic biological agents have either not been fully developed or validated in real-world settings, and are clearly deficient in applications for the detection and elimination of weaponized biological agents.

Traditional laboratory approaches to biological agent detection include microbial culturing, ELISA testing, and nucleic acid detection schemes, especially amplification methods such as the polymerase chain reaction (PCR). The last two approaches seek molecular evidence of agent components, such as characteristic immunological markers and genome sequences. A fourth broad approach relies upon the response of a surrogate host-such as cultivated cells from humans, animals, or plants for the determination of immediate risk. All of these methods are useful but they take far too much time to assist in the prevention or limitation of a serious natural outbreak of disease or even a minor use of a weaponized biological agent.

Accordingly, a need exists for the development of automated biosensor methods and devices for the detection and reporting of pathogens from monitoring sites is an important way of protecting populations from pathogenic biological agents. The methods of the current invention answer this need and also make the development of these biosensor antibodies cost effective.

### SUMMARY OF THE INVENTION

Briefly stated, the present invention relates generally to the production and purification of antibodies or modified antibodies that make them available for the detection of pathogenic agents of interest and/or chemical agents.

These and other objects which will be more readily apparent upon reading the following disclosure may be achieved by the present invention. Uses for recombinant biosensor antibodies include use for the detection of known Weapons of Mass Destruction (“WMD”) agents in particular biological agents including: anthrax, smallpox, botulism, ebola, hanta virus etc.

An objective of the current invention is to provide an antibody array that can be used to detect antibody expression profiles. According to a preferred methodology of the current invention several antibodies can be simultaneously examined with an array comprising a large number of immobilized antibodies put down in a pre-determined order. This then allows for multiple antibodies to recognize their corresponding antigens independently without cross reactions. When associated with known reporter mechanisms this interaction then allows for output data to indicate the type and breadth of a given biological agent/chemical agent threat.

An objective of the current invention is to provide an antibody array of the invention can also be used to detect antibody—protein interactions. We provide here the use of an antibody array in detection and identification of proteins that interact with specific proteins of interest.

### BRIEF DESCRIPTION OF THE DRAWINGS

FIG. 1 Shows a Flowchart of an Embodiment of the Process of Creating Cloned Animals through Nuclear Transfer.

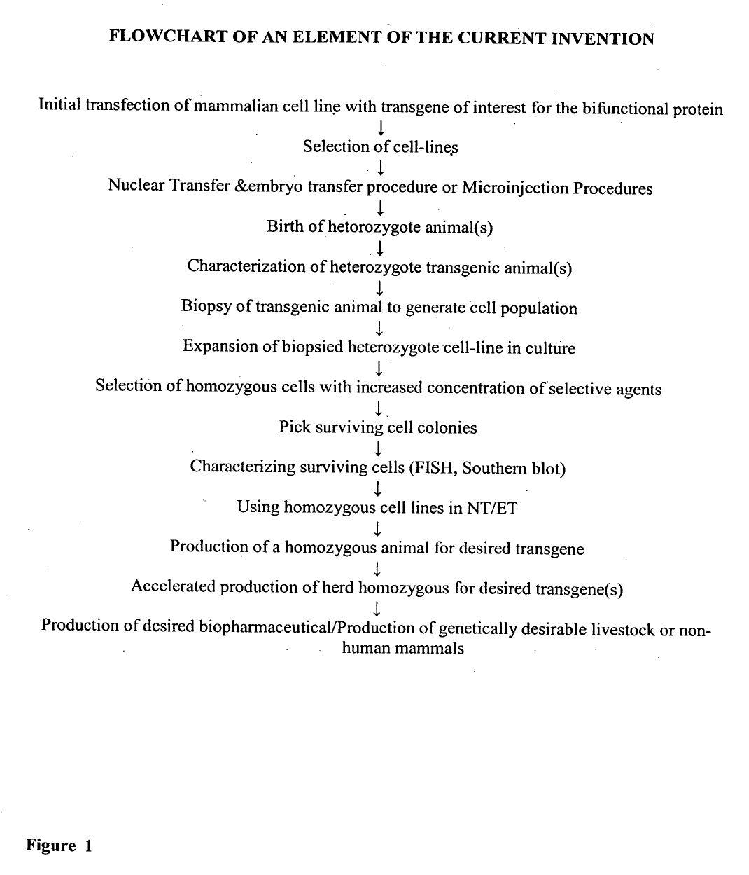

FIG. 2 Shows a Simple Schematic Representation of Biosensor Antibody with two identical antibody antigen/analyte binding sites. It is composed of four polypeptide chains-two identical heavy chains and two identical light chains. The two antigen-binding sites are identical, each formed by the N-terminal region of a light chain and the N-terminal region of a heavy chain. Both the tail (Fc) and hinge region are formed by the two heavy chains.

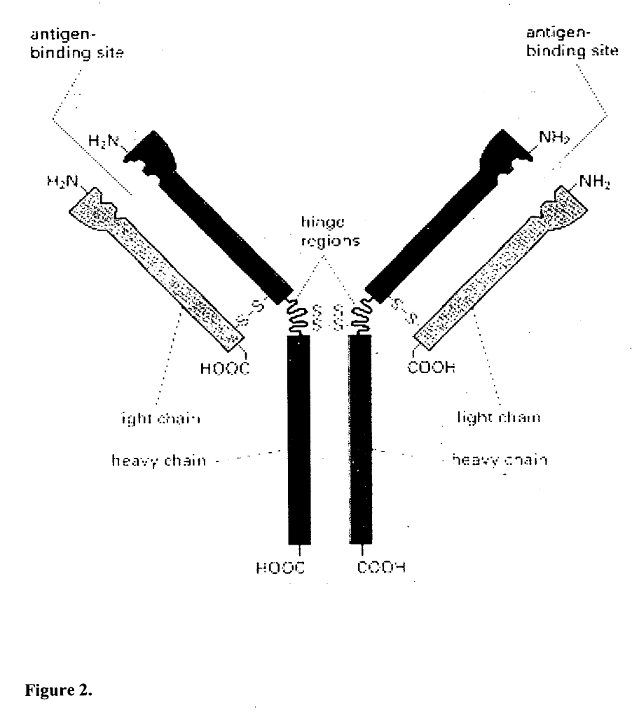

FIG. 3 Shows the Construction of a cDNA Vector for Biosensor Antibodies.

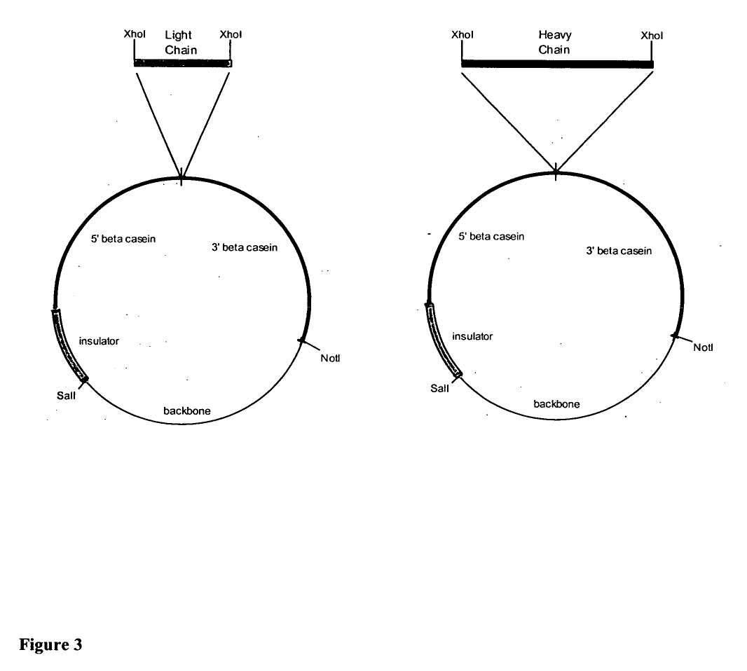

FIG. 4 Shows a Process for the Purification of Antibodies of Interest from a Milk Feedstream.

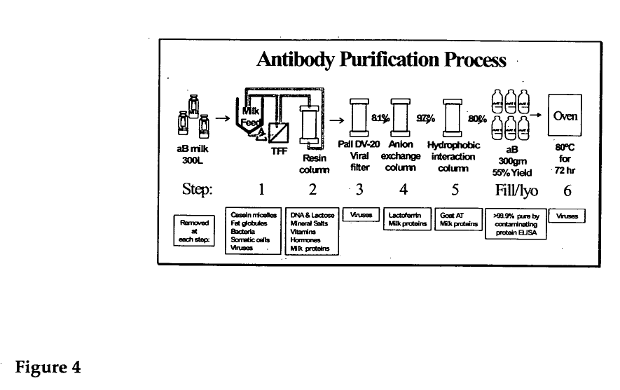

FIG. 5 Shows Embodiment of potential Biosensor Antibody Array of the Invention.

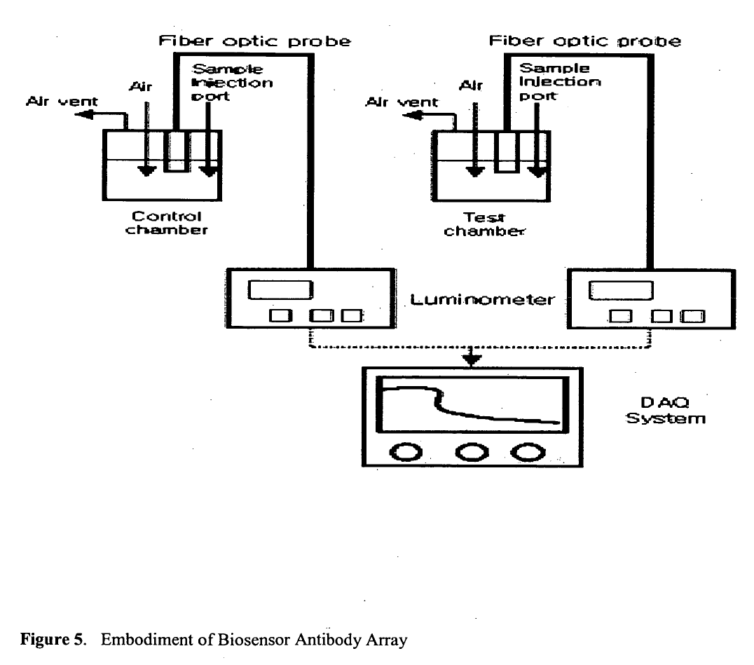

FIG. 6 Shows known and efficient peptide coupling reagents for solid support attachment of biosensor antibodies.

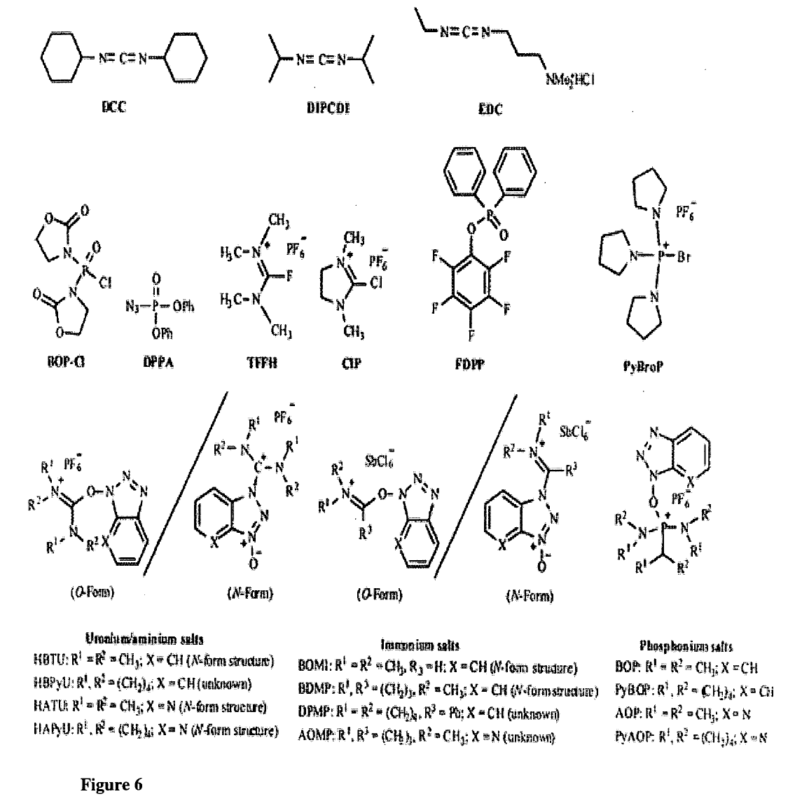

### DETAILED DESCRIPTION

The following abbreviations have designated meanings in the specification:

Abbreviation Key:
Somatic Cell Nuclear Transfer (SCNT)
Nuclear Transfer (NT)
Polymerase Chain Reaction (PCR)
Mean Fluorescence Intensity (MFI)
Rolling Circle Amplification (RCA)
Coefficient of Variation (CV)
R²: correlation coefficient (R²)
Lower Limit of Quantitation (LLQ)
Upper Limit of Quantitation (ULQ)
Minimal Detection Limits (MDL)
Standard Deviation (SD)

Explanation of Terms:
ABSORPTION: The process of an agent being drawn into a media through a surface (clothing, fabric, wood, etc.), much like a sponge and water.
ACETYLCHOLINE: A chemical compound formed from an acid and an alcohol, which causes muscles to contract and acts as a neurotransmitter. It is found in various organs and tissues of the body. It is rapidly broken down by an enzyme, cholinesterase.
ACETYLCHOLINESTERASE: An enzyme which inhibits the action of acetylcholine by breaking acetylcholine into its component parts. Nerve agents combine with acetylcholinesterase to prevent it from performing its inactivation of acetylcholine.
ADSORPTION: The process of an agent sticking to or becoming chemically attached to a surface.
AEROSOLS: A suspension or dispersion of small particles (solids or liquids) in a gaseous medium.
ANALYTE: Compound which is to be measured in an assay or biosensor application.
ANTIBODY AGENTS: Refers to antibodies, recombinant antibodies, calin, synthesized antibody fragments or single chain antibodies immobilized on the solid support of an antibody array.
ANTICHOLINERGIC: An agent or chemical that blocks or impedes the action of acetylcholine, such as the (also cholinolytic) antidote atropine.
ANTI-CHOLINESTERASE: A substance that blocks the action of cholinesterase (acetylcholinesterase) such as nerve agents.
ANTIDOTE: A substance that neutralizes toxic agents or their effects.
ATROPINE: An anticholinergic used as an antidote for nerve agents to counteract excessive amounts of acetylcholine. It also has other medicinal uses.
BIOLOGICAL FLUID: An aqueous solution produced by an organism, such as a mammal, bird, amphibian, or reptile, which contains antibodies that are secreted by cells that are bathed in the aqueous solution. Examples include: milk, urine, saliva, seminal fluid, vaginal fluid, synovial fluid, lymph fluid, amniotic fluid, blood, sweat, and tears; as well as an aqueous solution produced by a plant, including, for example, exudates and guttation fluid, xylem, phloem, resin, and nectar.
BLISTER AGENT: A chemical warfare agent which produces local irritation and damage to the skin (vesicant) and mucous membranes, pain and injury to the eyes, reddening and blistering of the skin, and when inhaled, damage to the respiratory tract.
BLOOD AGENT: A chemical warfare agent that is inhaled and absorbed into the blood. The blood carries the agent to all body tissues where it interferes with the tissue oxygenation process (ex: potassium cyanide).
CHEMICAL AGENT: Any chemical substance which is intended for use in military operations to kill, seriously injure, or incapacitate humans because of its physiological effects.
CHEMICAL WARFARE AGENT: A chemical substance, which, because of its physiological, psychological, or pharmacological effects, is intended for use in military operations to kill, seriously injure, or incapacitate humans (or animals) through its toxicological effects. Excluded are riot control agents, chemical herbicides, and smoke and flame agents.
CHOKING AGENTS: These agents exert their effects solely on the lungs and result in the irritation of the alveoli of the lungs. Agents cause the alveoli to constantly secrete watery fluid into the air sacs, which is called pulmonary edema. When a lethal amount of a choking agent is received, the air sacs become so flooded that the air cannot enter and the victim dies of anoxia (oxygen deficiency); also known as a dry land drowning.
CONCENTRATION: The amount of a chemical agent present in a unit volume of air, usually expressed in milligrams per cubic meter (mg/m3).
DILUTION FACTOR: Dilution of contaminated air with uncontaminated air in a general area, room or building for the purposes of health hazard or nuisance control, and/or for heating and cooling.
DOSAGE: The concentration of a chemical agent in the atmosphere (C) multiplied by the time (t) the concentration remains, expressed as mg-min/m. The dosage (Ct) received by a person depends upon how long he is exposed to the concentration. That is, the respiratory dosage in mg-min//m³ is equal to the time in minutes as individual is unmasked in an agent cloud multiplied by the concentration of the cloud.
ENZYME CONJUGATE: A synthetic compound produced by linking an enzyme to an antibody or part of an antibody which recognizes another molecule (e.g. antibody or antigen), to the animal in which it was raised.
ENCODING: Refers generally to the sequence information being present in a translatable form, usually operably linked to a promoter (e.g., a beta-casein or beta-lacto globulin promoter). A sequence is operably linked to a promoter when the functional promoter enhances transcription or expression of that sequence. An anti-sense strand is considered to also encode the sequence, since the same informational content is present in a readily accessible form, especially when linked to a sequence which promotes expression of the sense strand. The information is convertible using the standard, or a modified, genetic code.
EPITOPE: A region of an antigenic molecule which acts as a recognition site for an antibody. An epitope consists of a minimum of three amino acids but most stable epitopes are often between 5-7 amino acids.
EXPRESSION VECTOR: A genetically engineered plasmid or virus, derived from, for example, a bacteriophage, adenovirus, retrovirus, poxvirus, herpesvirus, or artificial chromosome, that is used to transfer transgenic antibody coding sequence, operably linked to a promoter, into a host cell, such that the encoded recombinant transgenic antibody is expressed within the host cell.
FUNCTIONAL ANTIBODIES: Antibodies which have a biological or other activity or use, similar to that seen when produced endogenously.
INCAPACITATING AGENT: An agent that produces physiological or mental effects, or both, that may persist for hours or days after exposure, rendering an individual incapable of performing his or her assigned duties.
LEADER SEQUENCE [or a “signal sequence”]: A nucleic acid sequence that encodes an antibody secretory signal, and, when operably linked to a downstream nucleic acid molecule encoding a transgenic antibody and directs secretion. The leader sequence may be the native human leader sequence, an artificially-derived leader, or may obtained from the same gene as the promoter used to direct transcription of the transgene coding sequence, or from another antibody that is normally secreted from a cell.
LETHAL CHEMICAL AGENT: An agent that may be used effectively in a field concentration to produce death.
LIQUID DOSAGE: The weight of a liquid agent received by a person on his skin is usually expressed as dosage in milligrams of contaminant per kilogram of body weight (mg/kg). This is equivalent to parts per million (ppm).
METHOD OF DISSEMINATION: The way a chemical agent or compound is finally released into the atmosphere.
MILK-PRODUCING CELL: A cell (e.g., a mammary epithelial cell) that secretes an antibody into milk.
MILK-SPECIFIC PROMOTER: A promoter that naturally directs expression of a gene in a cell that secretes a antibody into milk (e.g., a mammary epithelial cell) and includes, for example, the casein promoters, e.g., α-casein promoter (e.g., alpha S-1 casein promoter and alpha S2-casein promoter), β-casein promoter (e.g., the goat beta casein gene promoter (DiTullio, BIOTECHNOLOGY 10:74-77, 1992), γ-casein promoter, and ε-casein promoter; the whey acidic antibody (WAP) promoter (Gorton et al., BIOTECHNOLOGY 5: 1183-1187, 1987); the β-lactoglobulin promoter (Clark et al., BIOTECHNOLOGY 7: 487-492, 1989); and the α-lactalbumin promoter (Soulier et al., FEBS LETTS. 297:13, 1992). Also included are promoters that are specifically activated in mammary tissue and are thus useful in accordance with this invention, for example, the long terminal repeat (LTR) promoter of the mouse mammary tumor virus (MMTV).
NERVE AGENTS: Agents that effect the transmission of nerve impulses by reacting with the enzyme cholinesterase, permitting an accumulation of acetylcholine and continuous muscle stimulation. The muscles tire due to over-stimulation and begin to contract.
NUCLEAR TRANSFER: This refers to a method of cloning wherein the nucleus from a donor cell is transplanted into an enucleated oocyte.
OPERABLY LINKED: A gene and one or more regulatory sequences are connected in such a way as to permit gene expression when the appropriate molecules (e.g., transcriptional activator antibodies) are bound to the regulatory sequences.
ORGANOPHOSPHATE: A compound with a specific phosphate group which inhibits acetylcholinesterase. Organophosphates are used in chemical warfare and as an insecticide.
PERSISTENT AGENT: An agent that remains in the target area for longer periods of time. Hazards from both vapor and liquid may exist for hours, days, or in exceptional cases, weeks or months after dissemination of the agent. As a general rule, persistent agents duration will be greater than 12 hours.
POLYPEPTIDE: A polymer whose monomer linkages are usually but not necessarily peptide bonds, and whose side groups are usually but not necessarily selected from the naturally occurring amino acid side groups such as hydrogen (glycine), methyl (alanine), etc. Numerous unnatural polypeptide derivations, with modifications to both the amine linkages and side groups are known. Relatively small polypeptides are often termed peptides.
SOLUBILITY: The ability of a material to dissolve in water or another liquid.
TEAR AGENTS: Compounds that cause a large flow of tears and intense eye pain and irritation of the skin.
TOXICITY: The property a material possesses which enables it to injure the physiological mechanism of an organism by chemical means, with the maximum effect being incapacitation or death.
TRANSGENE: Any piece of a nucleic acid molecule that is inserted by artifice into a cell, or an ancestor thereof, and becomes part of the genome of the animal which develops from that cell. Such a transgene may include a gene which is partly or entirely exogenous (i.e., foreign) to the transgenic animal, or may represent a gene having identity to an endogenous gene of the animal.
TRANSGENIC ORGANISM: An organism into which genetic material from another organism has been experimentally transferred, so that the host acquires the genetic information of the transferred genes in its chromosomes in addition to that already in its genetic complement.
UNGULATE: Relating to a hoofed typically herbivorous quadruped mammal, including, without limitation, sheep, swine, goats, cattle and horses.
VECTOR: Means a plasmid, a phage DNA, or other DNA sequence that (1) is able to replicate in a host cell, (2) is able to transform a host cell, and (3) contains a marker suitable for identifying transformed cells.
VOLATILITY: With chemical agents, it refers to their ability to change from a liquid state into a gaseous state. That is, the ability of a material to evaporate.
VOMITING AGENT: Compounds that cause irritation of the upper respiratory tract and involuntary vomiting.

According to the present invention, there is provided a method for the production of the transgenic antibodies of interest for use in biosensor arrays. Preferably, the process comprises expressing in the milk of a transgenic non-human placental mammal one or more transgenic antibodies or antibody fragments useful for various biosensor applications.

Antibody Production

As stated above, the current invention pertains to the production of antibodies. In a preferred embodiment of the current invention the antibodies used for biosensor array applications are produced in the milk of a transgenic mammal, though other well-known methods exist in the prior art. These other methods can also be used for the production of antibodies for use in the methods of the current invention. Various aspects of the invention are also known in the prior art in regards to the methods of producing an antibody or fragments thereof. These fragments can also be preferably be produced in the milk of a transgenic mammal. Methods of producing a transgenic mammal whose somatic and germ cells include a modified antibody coding sequence are also known in the prior art. Use of nucleic acid sequences for expression for a variety of modified antibodies are also provided by the prior art.

Briefly then, as used herein, a “class” of antibodies refers to the five major isotypes of antibodies, including IgA, IgD, IgE, IgG, and IgM. A “subclass” of antibodies refers to the a subclassification of a given class of antibodies based on amino acid differences among members of the class, e.g., the class of antibodies designated IgG can be divided into the subclasses of, e.g., IgG1, IgG2, IgG3, and IgG4, and the class of antibodies designated as IgA can be divided into the subclasses of IgA1 and IgA2.

The term “antibody” refers to a protein comprising at least one, and preferably two, heavy (H) chain variable regions (abbreviated herein as VH), at least one and preferably two light (L) chain variable regions (abbreviated herein as VL), and at least one, preferably two heavy chain constant regions. The VH and VL regions can be further subdivided into regions of hypervariability, termed “complementarity determining regions” (“CDR”), interspersed with regions that are more conserved, termed “framework regions” (FR). The extent of the framework region and CDR's has been precisely defined (see, Kabat, E. A., et al. (1991) SEQUENCES OF PROTEINS OF IMMUNOLOGICAL INTEREST, (5^{th} Ed., U.S. Department of Health and Human Services, NIH Publication No. 91-3242); and, Chothia, C. et al. (1987) J. MOL. BIOL. 196:901-917, which are incorporated herein by reference). Each VH and VL is composed of three CDR's and four FRs, arranged from amino-terminus to carboxy-terminus in the following order: FR1, CDR1, FR2, CDR2, FR3, CDR3, FR4.

The antibodies of the invention can further include a light chain constant region, to thereby form a heavy and light immunoglobulin chains. In one embodiment, the antibody is a tetramer of two heavy immunoglobulin chains and two light immunoglobulin chains, wherein the heavy and light immunoglobulin chains are inter-connected by, e.g., disulfide bonds. The heavy chain constant region is comprised of three domains, CH1, CH2 and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system.

The antibodies of the invention can further include a hinge region, described in further detail below. As used herein, an “assembled” antibody is an antibody in which the heavy chains are associated with each other, e.g., interconnected by disulfide bonds. Each heavy chain hinge region includes at least one, and often several, cysteine residues. In the assembled antibody, the cysteine residues in the heavy chains are aligned so that disulfide bonds can be formed between the cysteine residues in the hinge regions covalently bonding the two heavy-light chain heterodimers together. Thus, fully assembled antibodies are bivalent in that they have two antigen binding sites. The term “antibody” (or “immunoglobulin”) as used herein, also refers to fragments of a full-length antibody, such as, e.g., a F(ab′)2 fragment, a bivalent fragment comprising two Fab fragments linked by a disulfide bridge at the hinge region. These antibody fragments are obtained using conventional techniques known to those with skill in the art, and the fragments are screened for utility in the same manner as are intact antibodies.

An “antigen-binding fragment” of an antibody (or “functional fragments”) refers to one or more portions of an antibody that retain the ability to specifically bind to an antigen. Examples of binding fragments encompassed within the term “antigen-binding fragment” of an antibody include one or more complementarities determining region (CDR).

As used herein, a “chimeric antibody heavy chain” refers to those antibody heavy chains having a portion of the antibody heavy chain, e.g., the variable region, at least 85%, preferably, 90%, 95%, 99% or more identical to a corresponding amino acid sequence in an antibody heavy chain from a particular species, or belonging to a particular antibody class or type, while the remaining segment of the antibody heavy chain (e.g., the constant region) being substantially identical to the corresponding amino acid sequence in another antibody molecule. For example, the heavy chain variable region has a sequence substantially identical to the heavy chain variable region of an antibody from one species (e.g., a “donor” antibody, e.g., a rodent antibody), while the constant region is substantially identical to the constant region of another species antibody (e.g., an “acceptor” antibody, e.g., a human antibody). The donor antibody can be an in vitro generated antibody, e.g., an antibody generated by phage display.

The term “humanized” or “CDR-grafted” light chain variable region refers to an antibody light chain comprising one or more CDR's, or having an amino acid sequence which differs by no more than 1 or 2 amino acid residues to a corresponding one or more CDR's from one species, or antibody class or type, e.g., a “donor” antibody (e.g., a non-human (usually a mouse or rat) immunoglobulin, or an in vitro generated immunoglobulin); and a framework region having an amino acid sequence about 85% or higher, preferably 90%, 95%, 99% or higher identical to a corresponding part of an acceptor antibody framework from a different species, or antibody class or type, e.g., a naturally-occurring immunoglobulin framework (e.g., a human framework) or a consensus framework. In some embodiments, the framework region includes at least about 60, and more preferably about 70 amino acid residues identical to those in the acceptor antibody light chain variable region framework, e.g., a naturally-occurring antibody framework (e.g., a human framework) or a consensus framework.

A “heterologous antibody” or “exogenous antibody” is an antibody that normally is not produced by the mammal, or is not normally produced in the mammary gland (e.g., an antibody only present in serum), or is produced in the mammary gland but the level of expression is augmented or enhanced in its production.

Any of the antibodies described herein, that is, chimeric, humanized or human antibodies, can include further modifications to their sequence. In this sense the sequences can be modified by addition, deletion or substitution, including a conservative substitution.

Insulator Sequences

The DNA constructs used to make the nucleic acid sequences of the antibodies of the invention will preferably employ one insulator sequence during their production. The terms “insulator”, “insulator sequence” and “insulator element” are used interchangeably herein. An insulator element is a control element which insulates the transcription of genes placed within its range of action but which does not perturb gene expression, either negatively or positively. Preferably, an insulator sequence is inserted on either side of the DNA sequence to be transcribed. For example, the insulator can be positioned about 200 bp to about 1 kb, 5′ from the promoter, and at least about 1 kb to 5 kb from the promoter, at the 3′ end of the gene of interest. The distance of the insulator sequence from the promoter and the 3′ end of the gene of interest can be determined by those skilled in the art, depending on the relative sizes of the gene of interest, the promoter and the enhancer used in the construct. In addition, more than one insulator sequence can be positioned 5′ from the promoter or at the 3′ end of the transgene. For example, two or more insulator sequences can be positioned 5′ from the promoter. The insulator or insulators at the 3′ end of the transgene can be positioned at the 3′ end of the gene of interest, or at the 3′end of a 3′ regulatory sequence, e.g., a 3′ untranslated region (UTR) or a 3′ flanking sequence.

A preferred insulator is a DNA segment which encompasses the 5′ end of the chicken β-globin locus and corresponds to the chicken 5′ constitutive hypersensitive site as described in PCT Publication 94/23046, the contents of which is incorporated herein by reference.

Purification of Proteins from Milk

A preparation, as used-herein, refers to two or more antibody molecules. In a preferred embodiment the antibody molecules of interest can be produced by one or more than one transgenic animal. Certain embodiments of the invention can also provide antibodies or kallikrines that vary in glycosylation profile for what is seen in vivo.

A “purified preparation”, “substantially pure preparation of antibodies”, or “isolated antibodies as used herein, refers to an antibody that is substantially free of material with which it occurs in the milk of a transgenic mammal. The antibody is also preferably separated from substances, e.g., gel matrix, e.g., polyacrylamide, which is used to purify it. In one embodiment, the language “substantially free” includes preparations of an antibody having less than about 30% (by dry weight) of non-antibody material (also referred to herein as a “milk impurity” or “milk component”), more preferably less than about 20% of non-antibody material, still more preferably less than about 10% of non-antibody material, and most preferably less than about 5% non-antibody material. Non-antibody material includes casein, lipids (e.g., soluble lipids and phospholipids), lactose and other small molecules (e.g., glucose, galactose), small peptides (e.g., microbial peptides and anti-microbial peptides) and other milk proteins (e.g., whey proteins such as β-lactoglobulin and α-lactalbumin, lactoferrin, and serum albumin). The antibodies preferably constitute at least 10, 20, 50, 70, 80 or 95% dry weight of the purified preparation. Preferably, the preparation contains: at least 1, 10, or 100 μg of the antibodies; at least 1, 10, or 100 mg of the antibodies. In addition, the purified preparation preferably contains about 70%, 75%, 80%, 85%, 90%, 95%, 98% assembled antibodies.

Antibodies (and fragments thereof) can be isolated from milk using standard protein purification methods known in the art. For example, the methods of Kutzko et al. (U.S. Pat. No. 6,268,487) can be utilized to purify antibodies and/or fragments of the present invention.

Milk proteins, or according to the current invention—antibodies of interest, must often be purified by a combination of processes. For example, raw milk can first be fractionated to remove fats, for example, by skimming, centrifugation, sedimentation (H. E. Swaisgood, Developments in Dairy Chemistry, in: CHEMISTRY OF MILK PROTEIN, Applied Science Publishers, NY, 1982), acid precipitation (U.S. Pat. No. 4,644,056) or enzymatic coagulation with rennin or chymotrypsin. Next, the major milk proteins may be fractionated into either a clear solution or a bulk precipitate from which the specific protein of interest may be readily purified. As another example, French Patent No.# 2,487,642 describes the isolation of milk proteins from skim milk or whey by membrane ultrafiltration in combination with exclusion chromatography or ion exchange chromatography. Whey is first produced by removing the casein by coagulation with rennet or lactic acid. U.S. Pat. No. 4,485,040 describes the isolation of an alpha-lactoglobulin-enriched product in the retentate from whey by two sequential ultrafiltration steps. U.S. Pat. No. 4,644,056 provides a method for purifying immunoglobulin from milk or colostrum by acid precipitation at pH 4.0-5.5, and sequential cross-flow filtration first on a membrane with 0.1-1.2 micrometer pore size to clarify the product pool and then on a membrane with a separation limit of 5-80 kd to concentrate it. U.S. Pat. No. 4,897,465 teaches the concentration and enrichment of a protein such as immunoglobulin from blood serum, egg yolks or whey by sequential ultrafiltration on metallic oxide membranes with a pH shift. Filtration is carried out first at a pH below the isoelectric point (pI) of the selected protein to remove bulk contaminants from the protein retentate, and next at a pH above the pI of the selected protein to retain impurities and pass the selected protein to the permeate. A different filtration concentration method is taught by European Patent No. EP 467 482 B1 in which defatted skim milk is reduced to pH 3-4, below the pI of the milk proteins, to solubilize both casein and whey proteins. Three successive rounds of ultrafiltration or diafiltration then concentrate the proteins to form a retentate containing 15-20% solids of which 90% is protein.

As another example, milk containing an antibody of interest can initially be clarified. A typical clarification protocol can include the following steps:
(a) diluting milk 2:1 with 2.0 M Arginine-HCl pH 5.5;
(b) spinning diluted sample in centrifuge for approximately 20 minutes at 4-8° C.;
(c) cooling samples for approximately 5 minutes on ice to allow fat sitting on top to solidify;
(d) removing fat pad by “popping” it off the top with a pipette tip; and,
(e) decanting of supernatant into a clean tube.

Further purification of proteins can be achieved using any method for protein purification known in the art, by methods as described above.

Obtaining Antibody Genes

According to the current invention antibody DNA sequences can be derived in several ways. One method provides for using mRNA derived from antibody-producing cells such as antigen-stimulated B lymphocytes from the spleen or peripheral blood. Hybridomas are also a source of mRNA that predominantly or exclusively encodes a single antibody. Because mRNAs have a polyadenylate (polyA) sequence at their 3′ end, they can be purified from the total RNA population by affinity chromatography on oligodeoxythymidylate-cellulose according to methods known in the prior art. A complementary DNA (cDNA) copy of the mRNA is then made using the enzyme reverse transcriptase. Commercial kits are also available that provide all the materials for mRNA isolation and preparation of cDNA. More straightforward methods include obtaining the recombinant antibody sequences of interest can be obtained by screening libraries of genomic material or reverse-translated messenger RNA derived from the animal of choice (such as cattle or mice), or through appropriate sequence databases such as NCBI, genbank, etc. These sequences along with the desired polypeptide sequence of the antibody of interest can then be cloned into an appropriate plasmid vector and amplified in a suitable host organism, usually E. coli. The DNA sequence encoding the peptide of choice can then be constructed, for example, by polymerase chain reaction amplification of a mixture of overlapping annealed oligonucleotides. The DNA sequence can then be inserted in a production system that would include in vitro cell culture systems or transgenic animal systems.

Antibody Engineering

Antibody engineering is the process of altering antibody structure and functional properties by recombinant DNA methods. According to the methods of the current invention once the DNA sequences of the variable regions are known, the amino acid sequence can be deduced. Methods of in vitro mutagenesis can then be applied to insert, delete, or change one or several amino acids, or to exchange entire variable domains as desired. Many laboratories worldwide are now using these techniques to produce antibodies that would be difficult or impossible to obtain from animals.

DNA Sequencing

After the desired antibody genes have been cloned and selected the DNA sequence is determined using chain termination sequencing methods that are now available in commercial kits (Maniatis et al., 1989). The sequencing requires oligonucleotide primers complementary to the 5′ ends of the region to be sequenced. As portions of the sequence are determined, additional primers may be needed to extend the sequence. Overlapping regions of sequence determined in separate runs are aligned to obtain the entire sequence. Both strands of the DNA are sequenced in order to verify the results. A frequently updated database of all reported antibody sequences has been compiled (Kabat et al., 1991) and is available. New antibody sequences can be analyzed by first comparing them to the most similar counterparts in known antibody sequence databases.

Changing the Structure of an Biosensor Antibody

A DNA sequence encoding a fusion protein sequence of the present invention is constructed using known recombinant DNA techniques to assemble separate DNA sequences encoding the first and second polypeptides into an appropriate expression vector. The 3′ end of a DNA sequence encoding the first polypeptide is ligated, with or without a peptide linker, to the 5′ end of a DNA sequence encoding the second polypeptide so that the reading frames of the sequences are in phase to permit mRNA translation of the two DNA sequences into a single fusion protein that retains the biological activity of both the first and the second polypeptides.

It also is possible, according to the current invention, to alter affinity and specificity or both by changing the relative orientations of VH and VL domains at their interface, lengthening or shortening particular CDRs to enlarge or shrink the binding pocket, increasing the flexibility of CDRs in the combining site, removing or re-spacing some of the side chains that form the combining site, or altering residues that do not contact antigen but help to form the combining site through CDR-CDR and CDR-framework interactions (Roberts et al., 1987). According to the current invention surface loops on the surface of the Fc portion can be changed, added, cyclized or lengthened to enhance adsorption to a surface and thereby improve the orientation of an antibody of interest to optimize sample capture. In principle other properties of the antibody structure antigen interactions can be changed by altering close-contact residues. That is, the antibodies of the invention can also be fused with other antibody molecules, toxins, or enzymes.

Building Amino Acid Linkages to Solid Supports

The solid support of the invention may be any solid material known to those of ordinary skill in the art to which the antigen may be attached. For example, the solid support may be a test well in a microtiter plate or a nitrocellulose or other membrane. Alternatively, the support may be a bead or disc, such as glass, gold, fiberglass, latex or a plastic material such as polystyrene or polyvinylchloride. The support may also be a magnetic particle or a fiber optic sensor, such as those disclosed, for example, in U.S. Pat. No. 5,359,681.

The polypeptides may be bound to the solid support using a variety of techniques known to those of ordinary skill in the art, which are amply described in the patent and scientific literature. In the context of the present invention, the term “bound” refers to both noncovalent association, such as adsorption, and covalent attachment (which may be a direct linkage between the antigen and functional groups on the support or may be a linkage by way of a cross-linking agent). Binding by covalent attachment through adding cyclized peptides to target antibodies is preferred.

In the case of adsorption, it may be achieved by contacting the polypeptide, in a suitable buffer, with the solid support for a suitable amount of time. The contact time varies with temperature, but is typically between about 1 hour and 1 day. In general, contacting a well of a plastic microtiter plate (such as polystyrene or polyvinylchloride) with an amount of polypeptide ranging from about 10 ng to about 1 μg, preferably about 100 ng, is sufficient to bind an adequate amount of antigen.

Covalent attachment of polypeptide to a solid support may generally be achieved by first reacting the support with a bifunctional reagent that will react with both the support and a functional group, such as a hydroxyl or amino group, on the polypeptide. For example, the polypeptide may be bound to supports having an appropriate polymer coating using benzoquinone or by condensation of an aldehyde group on the support with an amine and an active hydrogen on the polypeptide.

Cyclized Peptide Insertion into the Fc Region of Antibodies of Interest

Cyclization of peptides reduces the conformational freedom of these flexible, linear molecules, and often results in higher receptor binding affinities by reducing unfavorable entropic effects. Because of the more constrained structural framework, these agents are more selective in their affinity to specific receptor cavities and structurally offer better support. By the same reasoning, structurally constrained cyclic peptides confer greater stability against the action of proteolytic enzymes and can be more densely packed onto the surface of a selected support.

Methods for cyclization can be classified into “backbone to backbone” cyclization strategies through the formation of amide bond between the N-terminal and the C-terminal amino acid residues, and cyclizations involving the side chains of individual amino acids. The latter method includes: the formation of disulfide bridges between two w-thio amino acid residues (cysteine, homocysteine); the formation of lactam bridges between glutamic/aspartic acid and lysine residues; the formation of lactone or thiolactone bridges between amino acid residues containing carboxyl, hydroxyl or mercapto functional groups; the formation of thioether or ether bridges between the amino acids containing hydroxyl or mercapto functional groups; and, other special methods. According to the current invention, more recently developed general methods to effectively construct some of the aforementioned cyclic peptide derivatives can be used to aid in the anchoring of a biosensor antibody to a solid support.

Preferred Linking Strategies:

One is through attachment of an amino acid side chain to the solid support followed by on-resin cyclization and cleavage/deprotection (Strategy 1). Another approach utilizes electrophilic linkers that are sensitive to free amine bases, where cyclization affects cleavage from the solid support (Strategy 2). As for the third method, the C-terminal residue is linked via its alpha-nitrogen atom (Strategy 3). The first method was established in the early of 1990s and is becoming more widely used in cyclopeptide synthesis by attaching an amino acid side chain to solid supports such as Wang, HMPB-MBHA, PEG-PS and Sasrin resins. This strategy has been applied to aspartic and glutamic acids, lysine, serine, and tyrosine side chains in peptide syntheses with different C-terminal protecting groups. Strategy 2 relieves the limitation of having an appropriate side chain functionality at the C-terminus, but due to the prolonged reaction time needed for coupling, and the strong dependence of the coupling step on the peptide sequence, this method has not been widely adopted.

As for strategy 3, the C-terminal residue of the target peptide is anchored onto the acid cleavable resin via its alpha-nitrogen atom. Thus, this method does not require the presence of side chain functionality at the C-terminus and allows on-resin head-to-tail cyclization. This strategy, however, has suffered from substantial diketopiperazine formation. According to the current invention cyclized peptides consisting of less than 10 amino acids confer an added some degree of conformational rigidity when also bound to an antibody support structure. For larger cyclic peptides, additional constraints by introduction of rigid peptide backbone mimics or other conformational restrictions can further reduce conformational degrees of freedom. In this regard, the conformation ally constrained bicyclic antibodies of the invention provide a structurally balanced structure for specific biosensor antibodies.

In proteins and in many physiologically important eukaryotic peptides, the presence of intramolecular disulfide bonds provide major conformational controls towards the constraint and stabilization of structures. In a subset of natural product peptides, termed lantibiotics, there are cyclic sub-structures that contain thioether linkages spanning 4 to 7 amino acids. Lantibiotics, such as nisin, epidermin, and duramicycin, are produced by bacterial microorganisms. Many of these possess antimicrobial properties. The common building block in these peptides is lanthionine, an amino acid building block that consists of two alanines with their b-carbons bridged with a single sulfur atom. These thioether linked alanine subunits in lantibiotics are part of the cyclic peptide loops of various sizes in these natural products. According to the current invention these disulfide bonds or cyclized peptides may be added to antibodies or calins of interest to provide additional support, promote optimal orientation and optimal “packing” on a support.

According to one embodiment of the current invention the main advantage of thioether-cyclized peptide analogues over disulfide-linked constructs is the stability of the thioether to redox conditions. The creation of redox stable thioether linkages in rationally designed peptidomimetics is an attractive approach to restrict the conformational space and improve the biostability of such experimental agents.

Solid Supports

In the preferred embodiments, the agents immobilized on a solid support can be antibodies, recombinant antibodies, or modified antibodies. Antibodies are raised by immunizing animals (e.g., rabbit, mouse, rat, goat or chicken) with antigens (proteins or peptides). A large number of antibodies (monoclonal and polyclonal) are commercially available. Recombinant and modified antibodies are constructed by using recombinant DNA techniques.

In the preferred embodiments, the supports are either plates (glass or plastics), membranes made of nitrocellulose, nylon, or polyvinylidene difluoride (PVDF), or gold beads. Membranes are easier to handle and agents can be readily immobilized on them. Glass or plastic plates provide rigid support and are therefore necessary in some special applications.

According to an embodiment of the current invention agents are immobilized on a solid support directly or indirectly. Agents can be directly deposited at high density on a support, which can be as small as a microscopic slide. Similar technology was developed for a making high density DNA microarray (Shalon et al., GENOME RESEARCH, July 1996; 6(7): 639-45). Agents can also be immobilized indirectly on the support. For instance, antibody X or Y can be printed on a support. Agents (antibodies) are then immobilized on the support through their interactions with antibody X or Y. The advantage of this indirect method is that by engaging the constant regions of antibodies with antibody X or Y, the variable regions of the antibodies (antigen-binding domains) will be fully exposed to interact with antigens. Recombinant antibodies can be immobilized through the interaction between their tags and the ligands printed on the support. One most important characteristic of protein arrays utilizing visual cues or that is read visually is that all agents are immobilized at predetermined positions, so that each agent can be identified by its position or read as appropriate. Alternatively multiple array sections can be designed to report contact with antigens of interest through known methods (e.g., changes in weight, conductivity, fluorescence, chemical activity, etc.,).

Biosensor Diversity

According to the current invention the deciphering of the human genome sequence, elucidation of the complete genomes of many pathogens, and the development of new antibodies to those specific pathogenic agents provides new uses for substantial amounts of individual antibodies for various desirable biosensor applications. That is, in a preferred embodiment the current invention utilizes multiple antibodies directed towards a variety of pathogenic organisms or specific sensor molecules built into a biosensor antibody array detection device. In this fashion the current invention will provide unprecedented opportunities for antibody-based biosensor technologies to quickly detect and allow users to counter exposure to weaponized biological agents.

Biological agents of interest would include the following organisms, with antibodies of the invention directed against them and/or the biotoxins they produce: Bacillus Anthracis; Yersina Pestis; Yersina enterocolitica; Francisella Tularensis; Vibrio Cholerae; Vibrio parahemolyticus; Klebsiella species; Pseudomonas aeruginosa; Streptococci; Listeria; Cryptosporidium; Venezuelan Equine Encephalitis, Filoviridae (Ebola and Marburg viruses specifically); Brucella abortus; Brucella melitensis; Brucella suis; Nipah virus; Hendra virus (formerly called equine morbillivirus); Flaviviruses; Burkholderia mallei (formerly known as Pseudomonas mallei); Smallpox varieties or orthopoxvirus [two forms: variola major and variola minor]; Coxiella burnetii; Arenaviridae (Lassa fever, Argentine and Bolivian hemorrhagic fever), the Bunyaviridae (Hantavirus, Congo-Crimean hemorrhagic fever, Rift Valley fever, and Yellow fever) families; the Dengue hemorrhagic fever virus; Aeromonas sobria; Aeromonas hydrophila; Aeromonas caviae; Escherichia coli; Salmonella typhi; Salmonella paratyphi; Salmonella enteriditis; Salmonella cholera-suis; Salmonella typhimurium; Salmonella heidelberg; Shigella sonnei; Shigella flexneri; Shigella boydit; Shigella dysenteriae; Mycobacterium tuberculosis; Yersinia enterocolitca; Aeromonas hydrophila; Plesiomonas shigelloides; Campylobacteria jejuni; Campylobacreria coli; Bacteroides fragilis; Clostridia septicum; Clostridia perfingens; Clostridia botulinum; and Clostridia difficile.

Molecular toxins or chemicals that would be detected by embodiments of the biosensor array of the invention would include: ricin; sarin; soman, tabun; cyanogens; chloride and hydrogen chloride; oleoresin capsicum; arsene; chlorine, diphosgene; phosgene; distilled mustard, ethyldichloroarsine, mustard-lewisite mixture; nitrogen mustard; and, organophosphate pesticides; aflatoxin; Trichothecene mycotoxins, and the Staphylococcus enterotoxins A, B and C.

Agricultural bioterrorism causative agents, in addition to several of the above, would also include: foot-and-mouth disease, mad cow disease, swine fever and karnal bunt of wheat.

In a preferred embodiment the current invention relies on an array of various antibodies comprising a surveillance system for multiple aerosolized pathogenic agents of interest. In an additional embodiment of the current invention detection of pathogenic biological agents relies in part on the use of nucleic acid sequence databases for pathogen strain type identification and the use of that information for synthetic sequencing of an appropriate antigen carrying the detection target of interest. Once an antibody is raised and its nucleic acid sequence known it can be transgenically produced and made part of a specific biosensor array if a user determines that a given detection target should be included within a specific array. That is, the antibodies included in a given biosensor array can be engineered to focus on a specific set of pathogenic agents. Typically these would be biological agents deemed to be of the highest risk or likelihood at a given location or for a given role. Accordingly, the methods of the current invention provide for the selective, as well as, more efficient methods of production of selected antibodies modified to be useful for the detection of certain detrimental pathogens or antigens. This may be accomplished, according to the current invention, through the use of transgenically derived antibodies designed to detect pathogens of interest and then put together in certain combinations reflecting the profile of agents that users were aware of and want to be in a position to detect early.

The present invention, an antibody array for use in biosensor applications provides a powerful and quantitative tool for detection of a variety of antigens and/or protein sequences of interest. The antibody array of the invention is based on several principles. First, a protein can be recognized and identified unambiguously by specific molecules such as antibodies, recombinant proteins and small chemicals that can specifically interact with it. Second, an antibody or a small chemical can be immobilized on a solid support with the immobilized molecule retaining its ability interact in an antibody-protein binding. Agents (antibodies, recombinant proteins, and small chemicals) can be immobilized on solid supports such as glass plates, agarose beads, gold beads or PVDF membranes (LeGendre, 1990, BIOTECHNIQUES, Vol. 9, No. 6, p. 788-805). Third, many different antibody agents can be immobilized at different positions on a solid support without cross interactions among them, with supporting reporting mechanism means remaining intact and specific. This insures that each agent independently interacts with its respective detection target—typically a protein.

Weight

According to the current invention different biosensor antibody arrays can be made for different purposes or perceived threats. For instance, a typical WMD array could be made against a variety known biotoxins, viruses or small molecules developed for that purpose or thought to be available for that purpose. A separate array could be made to detect for multiple small molecule agents present in various Chemical Agents or Blistering Agents—such as sarin gas or mustard gas or for application in a specific location such as an urban environment or in a food processing center with the bioarray focusing on bacterial agents that could cause food spoilage or virsues indicating animals or produce affected by specific pathogens. In order to reveal the broad protein expression pattern in a source (e.g. a cell line), thousands of different antibodies are immobilized in a single support. The amount of antibodies immobilized can also be different, preferably in the range of nanogram to microgram. The number of different agents immobilized on one solid support varies depending on the particular applications envisioned for the needs of the users.

Also important is the concept that the protein sample can also be labeled by biotinylation in vitro. Biotinylated proteins trapped on the array will then be detected by avidin or streptavidin which strongly binds biotin. If avidin is conjugated with horseradish peroxidase or alkaline phosphatase, the captured protein can be visualized by enhanced chemical luminescence. The amount of proteins bound to each antibody represents the level of the specific protein in the sample. If a specific group of proteins are interested, they can be detected by agents which specifically recognize them. Other methods, like immunochemical staining, surface plasmon resonance, matrix-assisted laser desorption/ionization-time of flight, can also be used to detect the captured proteins and provide means to appropriately indicate the specific agents detected.

### Antibody Immobilization

### EXAMPLE 1

For exemplary purpose, we chose PVDF membrane as the support and manually deposited antibodies on it. After 10 minutes incubation, all antibodies became immobilized. The number of antibodies immobilized on the array varied depending on the applications and the amount of antibodies immobilized typically is in the range of 0.1 microgram to 1.0 microgram.

Single Antibodies for Detection of Variable Agents

Currently a large number of tests rely on a small number of specific antibodies or microbial genomic sequences. This reliance creates vulnerabilities—for example, with respect to pathogenic biological agents having modified antibody epitopes (binding sites) or sequences. Rather than relying on methods that target specific, known organisms, one would like to have detection methods that target groups of organisms (i.e., all members of these groups) and that can identify specific members of the group, including recognition of those that may not yet have been characterized. This would likely rely on consensus sequences and/or extremely well-preserved genetic sequences or sites among related groups of potential pathogenic organisms.

A further challenge is the need for highly sensitive systems, as some highly infectious pathogens require the inhalation of only 1 to 10 organisms to cause disease. In general, much greater attention is needed to translate basic laboratory research into field applications and clinical validation. Finally, because no test is perfect, it is important to be able to anticipate false-positive test results in a reliable and quantitative fashion. One potential strategy for minimizing the impact of false-positive test results is to create a system of multiple, parallel, independent technical platforms so as to avoid dependence on any one testing procedure. This requires crosscutting, interdisciplinary science (e.g., combining environmental microbiology, cell biology, biophysics, electronics, materials science and microfabrication, microfluidics, and bioinformatics/statistics).

Preferred Antibodies

Antibodies of the present invention include naturally purified products, products of chemical synthetic procedures, and products produced by recombinant techniques from a eukaryotic host, including, for example, yeast, higher plant, insect and mammalian cells. Depending upon the host employed in a recombinant production procedure, the antibody of the present invention can be glycosylated or can be non-glycosylated, with glycosylation being preferred, and glycosylation from a transgenic mammal being most preferred. Such methods are described in many standard laboratory manuals, such as Sambrook, supra, Sections 17.37-17.42; Ausubel, supra, Chapters 10, 12, 13, 16, 18 and 20, Colligan, ANTIBODY SCIENCE, supra, Chapters 12-14, all entirely incorporated herein by reference.

Biosensor Antibodies

The isolated antibodies and antibodies of the present invention comprise at least one antibody and/or antibody amino acid sequence disclosed or described herein encoded by any suitable polynucleotide, or any at least one isolated or prepared antibody. Preferably, the at least one antibody has at least one biosensor activity and the at least one antibody binds human biosensor antibody and, thereby partially or substantially modulates at least one structural or biological activity of at least one biosensor antibody.

As used herein, the term “biosensor antibody of the current invention” refers to a antibody as described herein that has at least one biosensor-dependent activity, such as 5-10,000%, of the activity of a known or other biosensor antibody or active portion thereof, preferably by at least about 10, 20, 30, 40, 50, 55, 60, 65, 70, 75, 80, 85, 90, 91, 92, 93, 94, 95, 96, 97, 98, 99, 100% or more, depending on the assay. The capacity of a biosensor antibody of the current invention to have at least one biosensor-dependent activity is preferably assessed by at least one suitable biosensor antibody or receptor assay, as described herein and/or as known in the art.

As used herein, the term “neutralizing antibody” refers to an antibody that can inhibit at least one biosensor-dependent activity by about 5-120%, preferably by at least about 10, 20, 30, 40, 50, 55, 60, 65, 70, 75, 80, 85, 90, 91, 92, 93, 94, 95, 96, 97, 98, 99, 100% or more depending on the assay. The capacity of a biosensor antibody of the current invention to inhibit a biosensor-dependent activity is preferably assessed by at least one suitable biosensor antibody or receptor assay, as described herein and/or as known in the art. An antibody of the invention can be of any class (IgG, IgA, IgM, IgE, IgD, etc.) or isotype and can comprise a kappa or lambda light chain. In one embodiment, the human antibody comprises an IgG heavy chain or defined fragment, for example, at least one of isotypes, IgG1, IgG2, IgG3 or IgG4. Antibodies of this type can be prepared by employing a transgenic mouse or other transgenic non-human mammal comprising at least one human light chain (e.g., IgG, IgA and IgM (e.g., γ1, γ2, γ3, γ4) transgenes as described herein and/or as known in the art. In another embodiment, a biosensor antibody human antibody comprises an IgG1 heavy chain and an IgG1 light chain.

At least one antibody of the invention binds at least one specified epitope specific to at least one biosensor antibody of the current invention, subunit, fragment, portion or any combination thereof. The at least one epitope can comprise at least one antibody binding region that comprises at least one portion of the antibody, which epitope can optionally comprise at least one portion of at least one extracellular, soluble, hydrophillic, external or cytoplasmic portion of the antibody.

The at least one antibody of the present invention can preferably comprise at least one antigen-binding region that comprises at least one human complementarity determining region (CDR1, CDR2 and CDR3) or variant of at least one heavy chain variable region and/or at least one human complementarity determining region (CDR1, CDR2 and CDR3) or variant of at least one light chain variable region. In a particular embodiment, the antibody and antibody can have an antigen-binding region that comprises at least a portion of at least one heavy chain (HC) CDR (i.e., HC CDR1, HC CDR2 and/or HC CDR3) having the amino acid sequence of the corresponding HC CDRs 1, 2 and/or 3. In another particular embodiment, the antibody or antigen-binding portion or variant can have at least one antigen-binding region that comprises at least a portion of at least one light chain (LC) CDR (i.e., LC CDR1, LC CDR2 and/or LC CDR3). In a preferred embodiment the three heavy chain CDRs and the three light chain CDRs of the antibody or antigen-binding fragment have the amino acid sequence of the corresponding CDR of at least one of biosensor antibody monoclonal antibody, as described herein. Such antibodies can be prepared by chemically joining together the various portions (e.g., CDRs, framework) of the antibody using conventional techniques, by preparing and expressing a (i.e., one or more) nucleic acid molecule that encodes the antibody using conventional techniques of recombinant DNA technology or by using any other suitable method.

The biosensor antibody of the current invention can comprise at least one of a heavy or light chain variable region having a defined amino acid sequence. For example, in a preferred embodiment, antibodies of the current invention comprises at least one of at least one heavy chain variable region; and/or at least one light chain variable region.

Antibody Arrays

Several novel truncated antibody constructs according to the current invention are possible, including single chain Fv-Fc dimers, minibodies which lack the antibody CH1 and CH2 domains, and maxibodies which lack the CH1 domain.

An antibody according to the present invention can include any antibody or peptide containing molecule that comprises at least a portion of an immunoglobulin molecule, such as but not limited to at least one complementarity determining region (CDR) (also termed the hypervariable region or HV) of a heavy or light chain variable region, or a ligand binding portion thereof, a heavy chain or light chain variable region, a heavy chain or light chain constant region, a framework region, or any portion thereof, wherein the antibody can be incorporated into an antibody of the present invention. An antibody of the invention can include or be derived from any mammal, such as but not limited to a human, a mouse, a rabbit, a rat, a rodent, a primate, or any combination thereof, and the like.

The present invention provides, in one aspect, isolated nucleic acid molecules comprising, complementary, or hybridizing to, a polynucleotide encoding specific biosensor antibody antibodies, comprising at least one specified sequence, domain, portion or variant thereof. The present invention further provides recombinant vectors comprising at least one biosensor antibody of the current invention or antibody encoding or complementary nucleic acid molecules, host cells containing such nucleic acids and/or recombinant vectors, as well as methods of making and/or using such antibody nucleic acids, vectors and/or host cells.

At least one antibody of the invention binds at least one specified epitope specific to at least one biosensor antibody of the current invention, subunit, fragment, portion or any combination thereof. The at least one epitope can comprise at least one antibody binding region that comprises at least one portion of said antibody, which epitope is preferably comprised of at least 1-5 amino acids of at least one portion thereof, such as but not limited to, at least one functional, extracellular, soluble, hydrophillic, external or cytoplasmic domain of said antibody, or any portion thereof.

Bispecific, heterospecific, heteroconjugate or similar antibodies can also be used that are monoclonal, preferably human or humanized, antibodies that have binding specificities for at least two different antigens. In the present case, one of the binding specificities is for at least one biosensor antibody of the current invention, the other one is for any other antigen. Methods for making bispecific antibodies are known in the art. Traditionally, the recombinant production of bispecific antibodies is based on the co-expression of two immunoglobulin heavy chain-light chain pairs, where the two heavy chains have different specificities (Milstein and Cuello, Nature 305:537 (1983)). Because of the random assortment of immunoglobulin heavy and light chains, these hybridomas (quadromas) produce a potential mixture of 10 different antibody molecules, of which only one has the correct bispecific structure. The purification of the correct molecule, which is usually done by affinity chromatography steps, is rather cumbersome, and the product yields are low. Similar procedures are disclosed, e.g., in WO 93/08829, U.S. Pat. Nos, 6,210,668, 6,193,967, 6,132,992, 6,106,833, 6,060,285, 6,037,453, 6,010,902, 5,989,530, 5,959,084, 5,959,083, 5,932,448, 5,833,985, 5,821,333, 5,807,706, 5,643,759, 5,601,819, 5,582,996, 5,496,549, 4,676,980, WO 91/00360, WO 92/00373, EP 03089, Traunecker et al., EMBO J. 10:3655 (1991), Suresh et al., METHODS IN ENZYMOLOGY 121:210 (1986), each entirely incorporated herein by reference.

Such antibodies optionally further affect a specific ligand, such as but not limited to where such antibody modulates, decreases, increases, antagonizes, angonizes, mitigates, aleviates, blocks, inhibits, abrogates and/or interferes with at least oneBiosensor antibody activity or binding, or with Biosensor antibody receptor activity or binding, in vitro, in situ and/or in vivo. As a non-limiting example, a suitable Biosensor antibody of the current invention, specified portion or variant of the present invention can bind at least oneBiosensor antibody, or specified portions, variants or domains thereof. A suitable biosensor antibody of the current invention, specified portion, or variant can also optionally affect at least one of antibody activity or function, such as but not limited to, RNA, DNA or antibody synthesis, antibody release, antibody receptor signaling, membrane antibody cleavage, antibody activity, antibody production and/or synthesis.

Biosensor antibodies useful in the methods and compositions of the present invention can optionally be characterized by high affinity binding to specific antigens.

Construction of Nucleic Acids

The isolated nucleic acids of the present invention can be made using (a) recombinant methods, (b) synthetic techniques, (c) purification techniques, or combinations thereof, as well-known in the art. The nucleic acids can conveniently comprise sequences in addition to a polynucleotide of the present invention. For example, a multi-cloning site comprising one or more endonuclease restriction sites can be inserted into the nucleic acid to aid in isolation of the polynucleotide. Also, translatable sequences can be inserted to aid in the isolation of the translated polynucleotide of the present invention. For example, a hexa-histidine marker sequence provides a convenient means to purify the antibodies of the present invention. The nucleic acid of the present invention—excluding the coding sequence—is optionally a vector, adapter, or linker for cloning and/or expression of a polynucleotide of the present invention.

Additional sequences can be added to such cloning and/or expression sequences to optimize their function in cloning and/or expression, to aid in isolation of the polynucleotide, or to improve the introduction of the polynucleotide into a cell. Use of cloning vectors, expression vectors, adapters, and linkers is well known in the art. (See, e.g., Ausubel, supra; or Sambrook, supra)

Recombinant Methods for Constructing Nucleic Acids

The isolated nucleic acid compositions of this invention, such as RNA, cDNA, genomic DNA, or any combination thereof, can be obtained from biological sources using any number of cloning methodologies known to those of skill in the art. In some embodiments, oligonucleotide probes that selectively hybridize, under stringent conditions, to the polynucleotides of the present invention are used to identify the desired sequence in a cDNA or genomic DNA library. The isolation of RNA, and construction of cDNA and genomic libraries, is well known to those of ordinary skill in the art. (See, e.g., Ausubel, supra; or Sambrook, supra)

Nucleic Acid Screening and Isolation Methods

A cDNA or genomic library can be screened using a probe based upon the sequence of a polynucleotide of the present invention, such as those disclosed herein. Probes can be used to hybridize with genomic DNA or cDNA sequences to isolate homologous genes in the same or different organisms. Those of skill in the art will appreciate that various degrees of stringency of hybridization can be employed in the assay; and either the hybridization or the wash medium can be stringent. As the conditions for hybridization become more stringent, there must be a greater degree of complementarity between the probe and the target for duplex formation to occur. The degree of stringency can be controlled by one or more of temperature, ionic strength, pH and the presence of a partially denaturing solvent such as formamide. For example, the stringency of hybridization is conveniently varied by changing the polarity of the reactant solution through, for example, manipulation of the concentration of formamide within the range of 0% to 50%. The degree of complementarity (sequence identity) required for detectable binding will vary in accordance with the stringency of the hybridization medium and/or wash medium. The degree of complementarity will optimally be 100%, or 70-100%, or any range or value therein. However, it should be understood that minor sequence variations in the probes and primers can be compensated for by reducing the stringency of the hybridization and/or wash medium.

Methods of amplification of RNA or DNA are well known in the art and can be used according to the present invention without undue experimentation, based on the teaching and guidance presented herein. In general, immunoassays are based upon the immunological reaction between proteins such as antibodies, antibody fragments, or even artificially generated elements simulating antibody binding sites such as peptides, templated polymers and the like (hereafter referred to as antibody recognition) and the substance for which they are specific, the ligand. Immunological reactions are characterized by their high specificity and accordingly, numerous schemes have been developed in order to take advantage of this characteristic. The goal is to identify a particular state with absolute specificity using as few assays as possible.

In the traditional heterogeneous forward assay, an antibody is immobilized on a solid phase such as microparticles, microtiter wells, paddles, and the like. The sample is then contacted with the immobilized antibody and the ligand binds if present in the sample. The bound substance is detected and quantitated by an entity associated directly or indirectly therewith. Such detectable entity include fluorescent molecules, chemiluminescent molecules, enzyme, isotopes, microparticles and the like. Many variants have been developed such as competition, indirect competition, and the like. Various methods are available to those skilled in the art for quantitating the amount of substance bound using these assays.

In addition to immunoassays, other diagnostic assays are available based upon the same demand for absolute specificity using wide range of recognition elements such as proteins (lectins, receptors, and the like), nucleic acids, carbohydrates, lipids and/or synthetic/engineered biomimetic compounds and the like. A wide range of basic techniques have also been developed including but not limited to microscopy, chromatography and electrophoresis in order to specifically identify diseases.

It is an object of this invention to provide an biosensor assay strategy for sample discrimination which relies upon an array of sensing elements with low specificity in order to increase the informational content of the diagnostic assay. The assay strategy is capable of discriminating subtle changes and thus allows early identification of changes in the state of health that can be of crucial importance. In some instances, the sensing elements used in conventional assays will be applicable. However, in most instances the specificity of these reagents will be too high to allow their use. Accordingly, new screening procedures will be developed in order to isolated reagents with appropriate combination of affinities and specificities and is an object of this invention, as well.

Synthetic Methods for Constructing Nucleic Acids

The isolated nucleic acids of the present invention can also be prepared by direct chemical synthesis by known methods (see, e.g., Ausubel, et al., supra). Chemical synthesis generally produces a single-stranded oligonucleotide, which can be converted into double-stranded DNA by hybridization with a complementary sequence, or by polymerization with a DNA polymerase using the single strand as a template. One of skill in the art will recognize that while chemical synthesis of DNA can be limited to sequences of about 100 or more bases, longer sequences can be obtained by the ligation of shorter sequences.

Recombinant Expression Cassettes

The present invention further provides recombinant expression cassettes comprising a nucleic acid of the present invention. A nucleic acid sequence of the present invention, for example a cDNA or a genomic sequence encoding an antibody of the present invention, can be used to construct a recombinant expression cassette that can be introduced into at least one desired host cell. A recombinant expression cassette will typically comprise a polynucleotide of the present invention operably linked to transcriptional initiation regulatory sequences that will direct the transcription of the polynucleotide in the intended host cell. Both heterologous and non-heterologous (i.e., endogenous) promoters can be employed to direct expression of the nucleic acids of the present invention.

In some embodiments, isolated nucleic acids that serve as promoter, enhancer, or other elements can be introduced in the appropriate position (upstream, downstream or in intron) of a non-heterologous form of a polynucleotide of the present invention so as to up or down regulate expression of a polynucleotide of the present invention. For example, endogenous promoters can be altered in vivo or in vitro by mutation, deletion and/or substitution.

Amino Acid Variation

A biosensor antibody of the current invention of the present invention can include one or more amino acid substitutions, deletions or additions, either from natural mutations or human manipulation, as specified herein.

Of course, the number of amino acid substitutions a skilled artisan would make depends on many factors, including those described above. Generally speaking, the number of amino acid substitutions, insertions or deletions for any given biosensor antibody of the current invention, fragment or variant will not be more than 40, 30, 20, 19, 18, 17, 16, 15, 14, 13, 12, 11, 10, 9, 8, 7, 6, 5, 4, 3, 2, 1, such as 1-30 or any range or value therein, as specified herein.

Amino acids in an biosensor antibody of the current invention of the present invention that are essential for function can be identified by methods known in the art, such as site-directed mutagenesis or alanine-scanning mutagenesis (e.g., Ausubel, supra, Chapters 8, 15; Cunningham and Wells, SCIENCE 244:1081-1085 (1989)). The latter procedure introduces single alanine mutations at every residue in the molecule. The resulting mutant molecules are then tested for biological activity, such as, but not limited to at least one biosensor neutralizing activity. Sites that are critical for antibody binding can also be identified by structural analysis such as crystallization, nuclear magnetic resonance or photoaffinity labeling (Smith, et al., J. MOL. BIOL. 224:899-904 (1992) and de Vos, et al., SCIENCE 255:306-312 (1992)).

Non-limiting CDRs or portions of biosensor antibodies or antibodies of the invention that can enhance or maintain at least one of the listed activities include, but are not limited to, any of the above polypeptides, further comprising at least one mutation corresponding to at least one substitution selected from the group consisting of at least one of extracellular, intracellular, soluble, at least 10 contiguous amino acids, and the like, extracellular, intracellular, soluble, at least 10 contiguous amino acids, and the like.

Non-limiting variants that can enhance or maintain at least one of the listed activities include, but are not limited to, any of the above polypeptides, further comprising at least one mutation corresponding to at least one substitution selected from the group consisting of any one or combination of those presented herein, e.g., but not limited to those presented in the current examples.

The antibodies and antibodies of the present invention, or specified variants thereof, can comprise any number of contiguous amino acid residues from an antibody of the present invention, wherein that number is selected from the group of integers consisting of from 10-100% of the number of contiguous residues in a biosensor antibody of the current invention or antibody. Optionally, this subsequence of contiguous amino acids is at least about 10, 20, 30, 40, 50, 60, 70, 80, 90, 100, 110, 120, 130, 140, 150, 160, 170, 180, 190, 200, 210, 220, 230, 240, 250 or more amino acids in length, or any range or value therein. Further, the number of such subsequences can be any integer selected from the group consisting of from 1 to 20, such as at least 2, 3, 4, or 5.

As those of skill will appreciate, the present invention includes at least one biologically active antibody or antibody of the present invention. Biologically active antibodies or antibodies have a specific activity at least 20%, 30%, or 40%, and preferably at least 50%, 60%, or 70%, and most preferably at least 80%, 90%, or 95%-1000% of that of the native (non-synthetic), endogenous or related and known antibody or antibody. Methods of assaying and quantifying measures of enzymatic activity and substrate specificity, are well known to those of skill in the art.

Preparation of Biotinylated Antibodies

Receptors are often labeled with biotin allowing the receptors to be immobilized to an avidin-coated support. Biotin labeling can be performed using the biotinylating enzyme, BirA (see, Schatz, BIOTECHNOLOGY 11:1138-43 (1993)).

Once expressed, collections of antibodies are purified from transgenic milk or culture media. Usually, antibody chains are expressed with signal sequences and are thus released to the culture media. However, if antibody chains are not naturally secreted by host cells, the antibody chains can be released by treatment with mild detergent. Antibody chains can then be purified by methods including ammonium sulfate precipitation, tangential flow filtration, high performance tangential flow filtration, affinity chromatography to immobilized target, column chromatography, gel electrophoresis and the like (see Scopes, in PROTEIN PURIFICATION (Springer-Verlag, N.Y., 1982)).

Purified antibodies are dialyzed against a minimum of 100 volumes of phosphate buffered saline (PBS), pH 7.4, for at least 4 hours. Antibodies are diluted to a final concentration of 2 mg/ml in PBS. A stock solution containing 40 mM of biotin-XX-NHS ester (Molecular Probes, Eugene, Oreg.) can be prepared in dimethylsulfoxide. The biotin-XX-NHS solution can be added to antibodies at a final concentration of 0.4 mM and reacted for 90 minutes at room temperature. Aminoethanesulfonic acid can be added to a final concentration of 20 mM and incubated for five minutes to quench remaining reactive groups. The biotinylated antibodies are dialyzed extensively to remove small molecules containing biotin from the antibodies.

According to the prior art, where physical adsorption of antibodies is concerned, the observed orientation has been essentially accidental, so that not all the adsorbed molecules could be expected to be capable of analyte capture. According to the prior art molecular orientation can some extent be controlled by buffer composition (e.g. relative pH and molecular charge), and the choice between available solid surfaces. However, since analyte binding sites are usually associated with functional groups, it may often be favorable to aim at hydrophobic adsorption, using a hydrophobic surface. According to the current invention more precise orientation control may be obtainable by covalent coupling or the addition of cyclized peptides. In general, if an available surface is not immediately suitable, it may be made so by coating with an appropriate precapture reagent.

According to the current invention it is desirable to immobilize antibodies in a single orientation, with the antigen-binding site positioned away from the surface and facing the capture solution, which will optimize the activities of immobilized biosensor antibodies and the performance of arrays. One available strategy involves the a site-specific introduction of biotin, which in turn provide a uniform orientation of the immobilized antibody. This overcomes the prior art limitation of the chemical biotinylation of antibodies, followed by their subsequent immobilization onto avidin- or streptavidin-coated surfaces. While these methods would likely allow biotinylated antibodies to retain their native conformation when immobilized, the antibodies are also often linked in multiple orientations relative to the surface.

For example, cyanosilane is an available coupling agent for direct binding of antibodies to silica supports. Also according to the current invention precise placement of the antibodies on a given support may be critically important for reporting purposes and may be accomplished through directed immobilization, direct spotting, or directed streptavidin-biotin attachment.

In addition, a variety of microarray substrates have been described in the prior art including: nylon membranes, plastic microwells, planar glass slides, gel-based arrays and beads in suspension arrays. Much effort has been expended in optimizing antibody attachment to the microarray substrate. Finally, various signal generation and signal enhancement strategies have been employed in antibody arrays, including colorimetry, radioactivity, fluorescence, chemiluminescence, quantum dots and other nanoparticles, enzyme-linked assays, resonance light scattering, tyramide signal amplification and rolling circle amplification. Each of these formats and procedures has distinct advantages and disadvantages, relating broadly to sensitivity, specificity, dynamic range, multiplexing capability, precision, throughput, and ease of use. In general, multiplexed microarray immunoassays are ambient analyte assays.

The anchored component may be immobilized by non-covalent or covalent attachments. Non-covalent attachment may be accomplished by simply coating the solid surface with a solution of the protein and drying. Alternatively, an immobilized antibody, preferably a monoclonal antibody, specific for the protein to be immobilized may be used to anchor the protein to the solid surface. The surfaces may be prepared in advance and stored.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously nonimmobilized component is pre-labeled, the detection of label immobilized on the surface indicates that complexes are formed. Where the previously nonimmobilized component is not pre-labeled, an indirect label can be used to detect complexes anchored on the surface; e.g., using a labeled antibody specific for the previously nonimmobilized component (the antibody, in turn, may be directly labeled or indirectly labeled with a labeled anti-Ig antibody). Alternatively, a reaction can be conducted in a liquid.

The type of array surface and the immobilization technique determines how effectively the target protein presents itself to the capture agent. The attachment must be such that the capture agent retains its conformation for binding and reproducibility of results and the capture event can be reported or visualized. Immobilization can be based on covalent or noncovalent interaction of the molecule with the surface. Noncovalent interactions include hydrophobic interactions, hydrogen bonding, van der Waals forces, electrostatic forces, or physical adsorption. These include the use of surfaces such as nitrocellulose or silane-coated glass slides and require no modifications to the molecule before attachment. However, since these interactions are weak, the molecules can get denatured or dislodged, thus causing loss of signal. The covalent attachment onto surfaces leads to molecules being arranged in a definite, orderly fashion and uses spacers and linkers to help minimize steric hindrances.

Alternative Substrate Binding Methods

The modified human antibodies and antibodies can be prepared using suitable methods, such as by reaction with one or more modifying agents. A “modifying agent” as the term is used herein, refers to a suitable organic group (e.g., hydrophilic polymer, a fatty acid, a fatty acid ester) that comprises an activating group. An “activating group” is a chemical moiety or functional group that can, under appropriate conditions, react with a second chemical group thereby forming a covalent bond between the modifying agent and the second chemical group. For example, amine-reactive activating groups include electrophilic groups such as tosylate, mesylate, halo (chloro, bromo, fluoro, iodo), N-hydroxysuccinimidyl esters (NHS), and the like. Activating groups that can react with thiols include, for example, maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hermanson, G. T., BIOCONJUGATE TECHNIQUES, in Academic Press: San Diego, Calif. (1996)). An activating group can be bonded directly to the organic group (e.g., hydrophilic polymer, fatty acid, fatty acid ester), or through a linker moiety, for example a divalent C₁-C₁₂ group wherein one or more carbon atoms can be replaced by a heteroatom such as oxygen, nitrogen or sulfur. Suitable linker moieties include, for example, tetraethylene glycol, —(CH₂)₃—, —NH—(CH₂)₆—NH—, —(CH₂)₂—NH—; and, —CH₂—O—CH₂—CH₂—O—CH₂—, CH₂—O—CH—NH—. Modifying agents that comprise a linker moiety can be produced, for example, by reacting a mono-Boc-alkyldiamine (e.g., mono-Boc-ethylenediamine, mono-Boc-diaminohexane)(“BOC”) with a fatty acid in the presence of 1-ethyl-3-(3-dimethylaminopropyl)carbodiimide (EDC) to form an amide bond between the free amine and the fatty acid carboxylate. The BOC protecting group can be removed from the product by treatment with trifluoroacetic acid (TFA) to expose a primary amine that can be coupled to another carboxylate as described, or can be reacted with maleic anhydride and the resulting product cyclized to produce an activated maleimido derivative of the fatty acid.

Antibody Arrays

The present invention also provides at least one biosensor antibody of the current invention or antibody composition comprising at least one, at least two, at, least three, at least four, at least five, at least six or more biosensor antibodies as described herein and/or as known in the art that are provided in a non-naturally occurring composition, mixture or form.

The biosensor antibody used in accordance with the present invention can be produced by recombinant means, including from mammalian cell or transgenic preparations, or can be purified from other biological sources, as described herein or as known in the art.

The range of at least oneBiosensor antibody of the current invention in at least one product of the present invention includes amounts yielding upon reconstitution, if in a wet/dry system, concentrations from about 1.0 ng/ml to about 1000 mg/ml, although lower and higher concentrations are operable and are dependent on the intended delivery vehicle, e.g., solution formulations will differ from transdermal patch, pulmonary, transmucosal, or osmotic or micro pump methods.

The range of at least one biosensor antibody of the current invention in at least one product of the present invention includes amounts yielding upon reconstitution, if in a wet/dry system, concentrations from about 1.0 μg/ml to about 1000 mg/ml, although lower and higher concentrations are operable and are dependent on the intended delivery vehicle, e.g., solution formulations will differ from transdermal patch, pulmonary, transmucosal, or osmotic or micro pump methods.

Ionizing Air and Collection of Particles

According to the purposes of the current invention electrostatic space chargers (negative air ionizers) have been found to reduce dust levels in poultry hatchers and housing facilities, thereby also diminishing airborne bacterial levels and the experimental transmission of Salmonella to chicks. Negative air ionization can also reduce S. enteritidis numbers in aerosols and on exposed surfaces. When applied to the current invention the ability of electrostatic charging to attract and hold bacteria or viruses borne on airborne particulate matter is critical to the success of this technology as a control strategy for monitoring the environmental presence or absence of pathogenic agents or chemical agents.

All electronic components except for the plug-in batteries are housed in a completely waterproof enclosure to allow disinfection by spraying after use. The device operates by applying a strong electrostatic field to draw charged airborne dust particles and aerosols onto the surface of antibody covered supports. As charged particles are drawn to the media, adjacent air is pulled in behind them-similar to the effect of air being pulled down with rain as it falls from a storm cloud-and the charged particles and associated airborne microorganisms are tightly bound to the media by electrostatic attraction. The electrostatic sampling device (with two plates each time) can be used to collect air samples for 20 min, 1 hr, or 3 hr. Once internalized ionized and purified water is added to provide an aqueous environment for testing with biosensor antibodies.

Method and Apparatus of the Current Invention

Description: Captures airborne bacteria, viruses, spores and other pathogens 0.5 microns and larger into a small volume of liquid. The collector uses cyclonic rotating impeller arms to impact airborne particles forcing them into a sample handling changer that is integrated with a portable aerosol collector. The captured liquid sample is then input into the biosensor antibody array. According to the current invention a portable system has been developed for rapid detection of aerosolized pathogenic biological agents. The system is based on coupling highly specific immunoassay technologies with ultra-sensitive reporting mechanisms. The apparatus of the current invention is optimized for detection of biological agents in liquid samples and in air by integration with portable aerosol collectors.
Purpose: Identification of the presence of biological compounds in aerosol using variable biosensor antibody arrays for detecting pathogenic biological organisms
Detection/Reporting: Within 15-20 minutes analytes detected, identified and reported.
Specificity: The current invention contemplates the use of a Biosensor array containing a variety of antibodies against identified pathogenic organisms or molecules. These antibodies are fixed to the surface of a biosensor antibody array and are exposed to a possible source of ligand. Typically two steps are involved in the process: first, the sample and reagent must be mixed manually, and, second, the sample must be applied to the presence or absence of the target antigen is detected by a small red dot that the user compares to a color chart.
Labeling: Antibody-conjugated horseradish peroxidase can be used as the enzymatic label and hydrogen peroxide and iodide ions used as substrates. Horseradish peroxidase catalyzes the oxidation of the iodide ions by hydrogen peroxide to giveiodine. This iodine is then electrochemically reduced at the working electrode surface with an uptake of two electrons. The flux of electrons out of the working electrode is measured in the form of a current signal. The magnitude of the current signal is proportional to the number of biological agents that have been captured and labeled.

The protein of interest is then selected by its unique property, i.e., interaction with an antibody. In some other applications such as immunoprecipitation and affinity purification, agents (e.g., antibodies, ligands) are covalently conjugated onto solid supports (e.g., agarose beads) through their primary amines, sulfhydryls or other reactive groups. In general, proteins retain their abilities of interacting with other proteins or ligands after immobilization.

We have designed a device (antibody array) which has many antibodies directed against specific pathogenic organisms immobilized on a solid support. Agents are immobilized in a predetermined order, i.e., each agent is immobilized at a specific position so that it can be identified by its unique position on the support. The device can capture and identify the specific proteins from a mixture (e.g., cell lysate). After capture and separation, the proteins can be further characterized. Therefore, the antibody array of the current invention makes it possible to study a wide variety of proteins in a single experiment by a large number of antibodies and/or recombinant proteins immobilized on a support.

Antibody array and the methods presented here have several significant advantages over the current methods. First, antibody array allows rapid detection of many proteins and thus makes it possible to compare protein expression profiles from different sources or those from the same source but under different conditions. Information on protein expression profile is very useful in identifying diagnostic and Biosensor targets. Second, antibody array makes it possible to detect posttranslational modifications of numerous proteins and provide a valuable tool to investigate protein and cellular regulations. Third, it can screen a large number of potential interactions directly; and it can detect interactions that take place only under certain conditions, e.g. phosphorylation. Antibody array is therefore useful for a variety of applications, particularly for revealing disease mechanisms, searching for diagnostic indicators and for identifying Biosensor targets. In addition, antibody array allows an individual user to access a large number of immobilized antibodies or recombinant proteins.

Biopolymers such as DNA, RNA, proteins or polypeptides, and polysaccharides can be directly activated using similar bi-functional silane compounds or other crosslinking reagents resulting in an immobilized biopolymer to a solid surface. This invention demonstrates that the target molecules to be arrayed are first modified so that they have binding affinity for solid surfaces without losing their probing abilities. Because the modification is a separate process, virtually any biological molecule can be modified and arrayed. Thus, a skilled artisan realizes that this invention is not limited to nucleic acids, but can be used for a spectrum of biological molecules.

Transgenic Goats & Cattle

The herds of pure- and mixed-breed certified scrapie-free Alpine, Saanen and Toggenburg dairy goats used as embryo, semen, cell and cell line donors for this study are maintained under Good Agricultural Practices. Similarly, cattle used should be maintained under Good Agricultural Practices and be certified to originate from a BSE-free herd. Transgenic founder goats are produced both by microinjection of the transgene into the pronucleus of a fertilized caprine egg or nuclear transfer technology.

Isolation of Caprine Fetal Somatic Cell Lines.

Primary caprine fetal fibroblast cell lines to be used as karyoplast donors are derived from 35- and 40-day fetuses. Fetuses are surgically removed and placed in equilibrated phosphate-buffered saline (PBS, Ca⁺⁺/Mg⁺⁺-free). Single cell suspensions are prepared by mincing fetal tissue exposed to 0.025% trypsin, 0.5 mM EDTA at 38° C. for 10 minutes. Cells are washed with fetal cell medium [equilibrated Medium-199 (M199, Gibco) with 10% fetal bovine serum (FBS) supplemented with nucleosides, 0.1 mM 2-mercaptoethanol, 2 mM L-glutamine and 1% penicillin/streptomycin (10,000 I.U. each/mL)], and are cultured in 25 cm flasks. A confluent monolayer of primary fetal cells can be harvested by trypsinization after 4 days of incubation and then maintained in culture or cryopreserved.

Preparation of Donor Cells for Embryo Reconstruction.

Transfected fetal somatic cells are seeded in 4-well plates with fetal cell medium and maintained in culture (5% CO₂, 39° C.). After 48 hours, the medium can be replaced with fresh low serum (0.5% FBS) fetal cell medium. The culture medium can be replaced with low serum fetal cell medium every 48 to 72 hours over the next 2-7 days following low serum medium, somatic cells (to be used as karyoplast donors) are harvested by trypsinization. The cells are re-suspended in equilibrated M199 with 10% FBS supplemented with 2 mM L-glutamine, 1% penicillin/streptomycin (10,000 I. U. each/mL) for at least 6 hours. The current experiments for the generation of desirable transgenic animals are preferably carried out with goat cells or mouse cells for the generation or goats or mice respectively but, according to the current invention, could be carried out with any mammalian cell line desired.

Oocyte Collection.

Oocyte donor does are synchronized and super ovulated as previously described (Ongeri, et al., 2001), and are mated over a 48-hour interval to fertile males for microinjection procedures and to vasectomized males for nuclear transfer procedures. After collection, fertilized embryos or unfertilized oocytes are cultured in equilibrated M199 with 10% FBS supplemented with 2 mM L-glutamine and 1% penicillin/streptomycin (10,000 I.U. each/mL).

Cytoplast Preparation and Enucleation.

All oocytes are treated with cytochalasin-B (Sigma, 5 μg/mL in SOF with 10% FBS) 15 to 30 minutes prior to enucleation. Metaphase-II stage oocytes are enucleated with a 25 to 30 μm glass pipette by aspirating the first polar body and adjacent cytoplasm surrounding the polar body (˜30% of the cytoplasm) to remove the metaphase plate. After enucleation, all oocytes are immediately reconstructed.

Nuclear Transfer and Reconstruction

Donor cell injection can be conducted in the same medium used for oocyte enucleation. One donor cell can be placed between the zona pellucida and the ooplasmic membrane using a glass pipet. The cell-oocyte couplets are incubated in SOF for 30 to 60 minutes before electrotransgenic and activation procedures. Reconstructed oocytes are equilibrated in transgenic buffer (300 mM mannitol, 0.05 mM CaCl₂, 0.1 mM MgSO₄, 1 mM K₂HPO₄, 0.1 mM glutathione, 0.1 mg/ml BSA) for 2 minutes. Electro-fusion and activation are conducted at room temperature, in a transgenic chamber with 2 stainless steel electrodes fashioned into a “transgenic slide” (500 μm gap; BTX-Genetronics, San Diego, Calif.) filled with transgenic medium.

Transgenic fusion can be performed using a transgenic slide. The transgenic slide can be placed inside a transgenic dish, and the dish can be flooded with a sufficient amount of transgenic buffer to cover the electrodes of the transgenic slide. Couplets are removed from the culture incubator and washed through transgenic buffer. Using a stereomicroscope, couplets are placed equidistant between the electrodes, with the karyoplast/cytoplast junction parallel to the electrodes. It should be noted that the voltage range applied to the couplets to promote activation and transgenic fusion can be from 1.0 kV/cm to 10.0 kV/cm. Preferably however, the initial single simultaneous transgenic and activation electrical pulse has a voltage range of 2.0 to 3.0 kV/cm, most preferably at 2.5 kV/cm, preferably for at least 20 μsec duration. This is applied to the cell couplet using a BTX ECM 2001 Electrocell Manipulator. The duration of the micropulse can vary from 10 to 80 μsec. After the process the treated couplet is typically transferred to a drop of fresh transgenic buffer. Transgenic treated couplets are washed through equilibrated SOF/FBS, then transferred to equilibrated SOF/FBS with or without cytochalasin-B. If cytocholasin-B is used its concentration can vary from 1 to 15 μg/mL, most preferably at 5 μg/mL. The couplets are incubated at 37-39° C. in a humidified gas chamber containing approximately 5% CO₂ in air. It should be noted that mannitol may be used in the place of cytocholasin-B throughout any of the protocols provided in the current disclosure (HEPES-buffered mannitol (0.3 mm) based medium with Ca⁺² and BSA).

Nuclear Transfer Embryo Culture and Transfer to Recipients.

Significant advances in nuclear transfer have occurred since the initial report of success in the sheep utilizing somatic cells (Wilmut et al., 1997). Many other species have since been cloned from somatic cells (Baguisi et al., 1999 and Cibelli et al., 1998) with varying degrees of success. Numerous other fetal and adult somatic tissue types (Zou et al., 2001 and Wells et al., 1999), as well as embryonic (Meng et al., 1997), have also been reported. The stage of cell cycle that the karyoplast is in at time of reconstruction has also been documented as critical in different laboratories methodologies (Kasinathan et al., BIOL. REPROD. 2001; Yong et al., 1998; and Kasinathan et al., NATURE BIOTECH. 2001).

All nuclear transfer embryos of the current invention are cultured in 50 μL droplets of SOF with 10% FBS overlaid with mineral oil. Embryo cultures are maintained in a humidified 39° C. incubator with 5% CO₂ for 48 hours before transfer of the embryos to recipient does. Recipient embryo transfer can be performed as previously described (Baguisi et al., 1999).

Similarly, known microinjection protocols can be utilized to produce a transgenic animal contemplated by the invention and capable of producing a desired antibody.

Pregnancy and Perinatal Care.

For goats, pregnancy can be determined by ultrasonography starting on day 25 after the first day of standing estrus. Does are evaluated weekly until day 75 of gestation, and once a month thereafter to assess fetal viability. For the pregnancy that continued beyond 152 days, parturition can be induced with 5 mg of PGF2μ (Lutalyse, Upjohn). Parturition occurred within 24 hours after treatment. Kids are removed from the dam immediately after birth, and received heat-treated colostrum within 1 hour after delivery. Time frames appropriate for other ungulates with regard to pregnancy and perinatal care (e.g., bovines) are known in the art.

Cloned Animals.

The present invention also includes a method of cloning a genetically engineered or transgenic mammal, by which a desired gene is inserted, removed or modified in the differentiated mammalian cell or cell nucleus prior to insertion of the differentiated mammalian cell or cell nucleus into the enucleated oocyte. Also provided by the present invention are mammals obtained according to the above method, and the offspring of those mammals. The present invention is preferably used for cloning caprines or bovines but could be used with any mammalian species. The present invention further provides for the use of nuclear transfer fetuses and nuclear transfer and chimeric offspring in the area of cell, tissue and organ transplantation.

Suitable mammalian sources for oocytes include goats, sheep, cows, pigs, rabbits, guinea pigs, mice, hamsters, rats, primates, etc. Preferably, the oocytes will be obtained from ungulates, and most preferably goats or cattle. Methods for isolation of oocytes are well known in the art. Essentially, this will comprise isolating oocytes from the ovaries or reproductive tract of a mammal, e.g., a goat. A readily available source of ungulate oocytes is from hormonally induced female animals.

For the successful use of techniques such as genetic engineering, nuclear transfer and cloning, oocytes may preferably be matured in vivo before these cells may be used as recipient cells for nuclear transfer, and before they can be fertilized by the sperm cell to develop into an embryo. Metaphase II stage oocytes, which have been matured in vivo, have been successfully used in nuclear transfer techniques. Essentially, mature metaphase II oocytes are collected surgically from either non-super ovulated or super ovulated animals several hours past the onset of estrus or past the injection of human chorionic gonadotropin (hCG) or similar hormone.

Moreover, it should be noted that the ability to modify animal genomes through transgenic technology offers new alternatives for the manufacture of recombinant proteins. The production of human recombinant pharmaceuticals in the milk of transgenic farm animals solves many of the problems associated with microbial bioreactors (e.g., lack of post-translational modifications, improper protein folding, high purification costs) or animal cell bioreactors (e.g., high capital costs, expensive culture media, low yields). The current invention enables the use of transgenic production of biopharmaceuticals, transgenic proteins, plasma proteins, and other molecules of interest in the milk or other bodily fluid (i.e., urine or blood) of transgenic animals hemizygous for a desired gene.

The use of living organisms as the production process means that all of the material produced will be identical in amino-acid sequence to the natural product. In terms of basic amino acid structures this means that only L-optical isomers, having the natural configuration, will be present in the product. Also the number of wrong sequences will be negligible because of the high fidelity of biological synthesis compared to chemical routes, in which the relative inefficiency of coupling reactions will always produce failed sequences. The absence of side reactions is also an important consideration with further modification reactions such as carboxy-terminal amidation. Again, the enzymes operating in vivo give a high degree of fidelity and stereospecificity which cannot be matched by chemical methods. Finally the production of a transgenic antibody of interest in a biological fluid means that low-level contaminants remaining in the final product are likely to be far less toxic than those originating from a chemical reactor.

As previously mentioned, expression levels of several grams per liter of caprine or bovine milk are well within the reach of existing transgenic animal technology. Such levels should also be achievable for the recombinant antibodies contemplated by the current invention.

After amplification of the vector, the DNA construct would be excised with the appropriate 5′ and 3′ control sequences, purified away from the remains of the vector and used to produce transgenic animals that have integrated into their genome the desired transgenic antibody. Conversely, with some vectors, such as yeast artificial chromosomes (YACs), it is not necessary to remove the assembled construct from the vector; in such cases the amplified vector may be used directly to make transgenic animals. In this case refers to the presence of a first polypeptide encoded by enough of a protein nucleic acid sequence to retain its biological activity, this first polypeptide is then joined to a the coding sequence for a second polypeptide also containing enough of a polypeptide sequence of a protein to retain its physiological activity. The coding sequence being operatively linked to a control sequence which enables the coding sequence to be expressed in the milk of a transgenic non-human placental mammal.

A DNA sequence which is suitable for directing production to the milk of transgenic animals carries a 5′-promoter region derived from a naturally-derived milk protein and is consequently under the control of hormonal and tissue-specific factors. Such a promoter should therefore be most active in lactating mammary tissue. According to the current invention the promoter so utilized can be followed by a DNA sequence directing the production of a protein leader sequence which would direct the secretion of the transgenic protein across the mammary epithelium into the milk. At the other end of the transgenic protein construct a suitable 3′-sequence, preferably also derived from a naturally secreted milk protein, may be added to improve stability of mRNA. An example of suitable control sequences for the production of proteins in the milk of transgenic animals are those from the caprine beta casein promoter.

The production of transgenic animals can now be performed using a variety of methods. The methods preferred by the current invention is microinjection or nuclear transfer.

Milk Specific Promoters.

The transcriptional promoters useful in practicing the present invention are those promoters that are preferentially activated in mammary epithelial cells, including promoters that control the genes encoding milk proteins such as caseins, beta-lacto globulin (Clark et al., (1989) BIO/TECHNOLOGY 7: 487-492), whey acid protein (Gorton et al. (1987) BIO/TECHNOLOGY 5: 1183-1187), and lactalbumin (Soulier et al., (1992) FEBS LETTS. 297: 13). Casein promoters may be derived from the alpha, beta, gamma or kappa casein genes of any mammalian species; a preferred promoter is derived from the goat beta casein gene (DiTullio, (1992) BIO/TECHNOLOGY 10:74-77). The milk-specific protein promoter or the promoters that are specifically activated in mammary tissue may be derived from either cDNA or genomic sequences. Preferably, they are genomic in origin.

DNA sequence information is available for all of the mammary gland specific genes listed above, in at least one, and often several organisms. See, e.g., Richards et al., J. BIOL. CHEM. 256, 526-532 (1981) (α-lactalbumin rat); Campbell et al., NUCLEIC ACIDS RES. 12, 8685-8697 (1984) (rat WAP); Jones et al., J. BIOL. CHEM. 260, 7042-7050 (1985) (rat β-casein); Yu-Lee & Rosen, J. BIOL. CHEM. 258, 10794-10804 (1983) (rat γ-casein); Hall, BIOCHEM. J. 242, 735-742 (1987) (α-lactalbumin human); Stewart, NUCLEIC ACIDS RES. 12, 389 (1984) (bovine αs1 and κ casein cDNAs); Gorodetsky et al., GENE 66, 87-96 (1988) (bovine β casein); Alexander et al., EUR. J. BIOCHEM. 178, 395-401 (1988) (bovine κ casein); Brignon et al., FEBS LETT. 188, 48-55 (1977) (bovine αS2 casein); Jamieson et al., GENE 61, 85-90 (1987), Ivanov et al., BIOL. CHEM. Hoppe-Seyler 369, 425-429 (1988), Alexander et al., NUCLEIC ACIDS RES. 17, 6739 (1989) (bovine β lactoglobulin); Vilotte et al., BIOCHIMIE 69, 609-620 (1987) (bovine α-lactalbumin). The structure and function of the various milk protein genes are reviewed by Mercier & Vilotte, J. DAIRY SCI. 76, 3079-3098 (1993) (incorporated by reference in its entirety for all purposes). To the extent that additional sequence data might be required, sequences flanking the regions already obtained could be readily cloned using the existing sequences as probes. Mammary-gland specific regulatory sequences from different organisms are likewise obtained by screening libraries from such organisms using known cognate nucleotide sequences, or antibodies to cognate proteins as probes.

Signal Sequences

Among the signal sequences that are useful in accordance with this invention are milk-specific signal sequences or other signal sequences which result in the secretion of eukaryotic or prokaryotic proteins. Preferably, the signal sequence is selected from milk-specific signal sequences, i.e., it is from a gene which encodes a product secreted into milk. Most preferably, the milk-specific signal sequence is related to the milk-specific promoter used in the expression system of this invention. The size of the signal sequence is not critical for this invention. All that is required is that the sequence be of a sufficient size to effect secretion of the desired recombinant protein, e.g., in the mammary tissue. For example, signal sequences from genes coding for caseins, e.g., alpha, beta, gamma or kappa caseins, beta lactoglobulin, whey acid protein, and lactalbumin are useful in the present invention. The preferred signal sequence is the goat β-casein signal sequence.

Signal sequences from other secreted proteins, e.g., proteins secreted by liver cells, kidney cell, or pancreatic cells can also be used.

Amino-Terminal Regions of Secreted Proteins.

The efficacy with which a non-secreted protein is secreted can be enhanced by inclusion in the protein to be secreted all or part of the coding sequence of a protein which is normally secreted. Preferably the entire sequence of the protein which is normally secreted is not included in the sequence of the protein but rather only a portion of the amino terminal end of the protein which is normally secreted. For example, a protein which is not normally secreted is fused (usually at its amino terminal end) to an amino terminal portion of a protein which is normally secreted.

Preferably, the protein which is normally secreted is a protein which is normally secreted in milk. Such proteins include proteins secreted by mammary epithelial cells, milk proteins such as caseins, beta lacto globulin, whey acid protein, and lactalbumin. Casein proteins include alpha, beta, gamma or kappa casein genes of any mammalian species. A preferred protein is beta casein, e.g., goat beta casein. The sequences which encode the secreted protein can be derived from either cDNA or genomic sequences. Preferably, they are genomic in origin, and include one or more introns.

DNA Constructs

The expression system or construct, described herein, can also include a 3′ untranslated region downstream of the DNA sequence coding for the non-secreted protein. This region apparently stabilizes the RNA transcript of the expression system and thus increases the yield of desired protein from the expression system. Among the 3′ untranslated regions useful in the constructs of this invention are sequences that provide a poly A signal. Such sequences may be derived, e.g., from the SV40 small t antigen, the casein 3′ untranslated region or other 3′ untranslated sequences well known in the art. Preferably, the 3′ untranslated region is derived from a milk specific protein. The length of the 3′ untranslated region is not critical but the stabilizing effect of its poly A transcript appears important in stabilizing the RNA of the expression sequence.

Optionally, the expression system or construct includes a 5′ untranslated region between the promoter and the DNA sequence encoding the signal sequence. Such untranslated regions can be from the same control region from which promoter is taken or can be from a different gene, e.g., they may be derived from other synthetic, semi-synthetic or natural sources. Again their specific length is not critical, however, they appear to be useful in improving the level of expression.

The construct can also include about 10%, 20%, 30%, or more of the N-terminal coding region of the gene preferentially expressed in mammary epithelial cells. For example, the N-terminal coding region can correspond to the promoter used, e.g., a goat β-casein N-terminal coding region.

The above-described expression systems may be prepared using methods well known in the art. For example, various ligation techniques employing conventional linkers, restriction sites etc. may be used to good effect. Preferably, the expression systems of this invention are prepared as part of larger plasmids. Such preparation allows the cloning and selection of the correct constructions in an efficient manner as is well known in the art. Most preferably, the expression systems of this invention are located between convenient restriction sites on the plasmid so that they can be easily isolated from the remaining plasmid sequences for incorporation into the desired mammal.

Prior art methods often include making a construct and testing it for the ability to produce a product in cultured cells prior to placing the construct in a transgenic animal. Surprisingly, the inventors have found that such a protocol may not be of predictive value in determining if a normally non-secreted protein can be secreted, e.g., in the milk of a transgenic animal. Therefore, it may be desirable to test constructs directly in transgenic animals, e.g., transgenic mice, as some constructs which fail to be secreted in CHO cells are secreted into the milk of transgenic animals.

Transgenic Mammals.

Preferably, the DNA constructs of the invention are introduced into the germ-line of a mammal. For example, one or several copies of the construct may be incorporated into the genome of a mammalian embryo by standard transgenic techniques known in the art.

Any non-human mammal can be usefully employed in this invention. Mammals are defined herein as all animals, excluding humans, which have mammary glands and produce milk. Preferably, mammals that produce large volumes of milk and have long lactating periods are preferred. Preferred mammals are cows, sheep, goats, mice, oxen, camels and pigs. Of course, each of these mammals may not be as effective as the others with respect to any given expression sequence of this invention. For example, a particular milk-specific promoter or signal sequence may be more effective in one mammal than in others. However, one of skill in the art may easily make such choices by following the teachings of this invention.

In an exemplary embodiment of the current invention, a transgenic non-human animal is produced by introducing a transgene into the germline of the non-human animal. Transgenes can be introduced into embryonal target cells at various developmental stages. Different methods are used depending on the stage of development of the embryonal target cell. The specific line(s) of any animal used should, if possible, be selected for general good health, good embryo yields, good pronuclear visibility in the embryo, and good reproductive fitness.

The litters of transgenic mammals may be assayed after birth for the incorporation of the construct into the genome of the offspring. Preferably, this assay is accomplished by hybridizing a probe corresponding to the DNA sequence coding for the desired recombinant protein product or a segment thereof onto chromosomal material from the progeny. Those mammalian progeny found to contain at least one copy of the construct in their genome are grown to maturity. The female species of these progeny will produce the desired protein in or along with their milk. Alternatively, the transgenic mammals may be bred to produce other transgenic progeny useful in producing the desired proteins in their milk.

In accordance with the methods of the current invention for transgenic animals a transgenic primary cell line (from either caprine, bovine, ovine, porcine or any other non-human vertebrate origin) suitable for somatic cell nuclear transfer is created by transfection of the transgenic protein nucleic acid construct of interest (for example, a mammary gland-specific transgene(s) targeting expression of a transgenic protein to the mammary gland). The transgene construct can either contain a selection marker (such as neomycin, kanamycin, tetracycline, puromycin, zeocin, hygromycin or any other selectable marker) or be co-transfected with a cassette able to express the selection marker in cell culture.

Transgenic females may be tested for protein secretion into milk, using any of the assay techniques that are standard in the art (e.g., Western blots or enzymatic assays).

The invention provides expression vectors containing a nucleic acid sequence described herein, operably linked to at least one regulatory sequence. Many such vectors are commercially available, and other suitable vectors can be readily prepared by the skilled artisan. “Operably linked” or “operatively linked” is intended to mean that the nucleic acid molecule is linked to a regulatory sequence in a manner which allows expression of the nucleic acid sequence by a host organism. Regulatory sequences are art recognized and are selected to produce the encoded polypeptide or protein. Accordingly, the term “regulatory sequence” includes promoters, enhancers, and other expression control elements which are described in Goeddel, GENE EXPRESSION TECHNOLOGY: METHODS IN ENZYMOLOGY 185, (Academic Press, San Diego, Calif. (1990)). For example, the native regulatory sequences or regulatory sequences native to the transformed host cell can be employed.

It should be understood that the design of the expression vector may depend on such factors as the choice of the host cell to be transformed and/or the type of protein desired to be expressed. For instance, the polypeptides of the present invention can be produced by ligating the cloned gene, or a portion thereof, into a vector suitable for expression in either prokaryotic cells, eukaryotic cells or both. (A LABORATORY MANUAL, 2nd Ed., ed. Sambrook et al. (Cold Spring Harbor Laboratory Press, 1989) Chapters 16 and 17)).

Following selection of colonies recombinant for the desired nucleic acid construct, cells are isolated and expanded, with aliquots frozen for long-term preservation according to procedures known in the field. The selected transgenic cell-lines can be characterized using standard molecular biology methods (PCR, Southern blotting, FISH). Cell lines carrying nucleic acid constructs of the obesity related transgenic protein of interest, of the appropriate copy number, generally with a single integration site (although the same technique could be used with multiple integration sites) can then be used as karyoplast donors in a somatic cell nuclear transfer protocol known in the art. Following nuclear transfer, and embryo transfer to a recipient animal, and gestation, live transgenic offspring are obtained.

Typically this transgenic offspring carries only one transgene integration on a specific chromosome, the other homologous chromosome not carrying an integration in the same site. Hence the transgenic offspring is hemizygous for the transgene, maintaining the current need for at least two successive breeding cycles to generate a homozygous transgenic animal.

Animal Promoters

Useful promoters for the expression of a target protein the mammary tissue include promoters that naturally drive the expression of mammary-specific polypeptides, such as milk proteins. These include, e.g., promoters that naturally direct expression of whey acidic protein (WAP), alpha S1-casein, alpha S2-casein, beta-casein, kappa-casein, beta-lactoglobulin, alpha-lactalbumin (see, e.g., Drohan et al., U.S. Pat. No. 5,589,604; Meade et al., U.S. Pat. No. 4,873,316; and Karatzas et al., U.S. Pat. No. 5,780,009), and others described in U.S. Pat. No. 5,750,172. Whey acidic protein (WAP; Genbank Accession No. X01153), the major whey protein in rodents, is expressed at high levels exclusively in the mammary gland during late pregnancy and lactation (Hobbs et al., J. BIOL. CHEM. 257:3598-3605, 1982). For additional information on desired mammary gland-specific promoters, see, e.g., Richards et al., J. BIOL. CHEM. 256:526-532, 1981 (α-lactalbumin rat); Campbell et al., NUCLEIC ACIDS RES. 12:8685-8697, 1984 (rat WAP); Jones et al., J. BIOL. CHEM. 260:7042-7050, 1985 (rat β-casein); Yu-Lee & Rosen, J. BIOL. CHEM. 258:10794-10804, 1983 (rat γ-casein); Hall, BIOCHEM. J. 242:735-742, 1987 (human α-lactalbumin); Stewart, NUCLEIC ACIDS RES. 12:3895-3907, 1984 (bovine α-s1 and κ-casein cDNAs); Gorodetsky et al., GENE 66:87-96, 1988 (bovine β-casein); Alexander et al., EUR. J. BIOCHEM. 178:395-401, 1988 (bovine κ-casein); Brignon et al., FEBS LETT. 188:48-55, 1977 (bovine α-S2 casein); Jamieson et al., GENE 61:85-90, 1987, Ivanov et al., BIOL. CHEM. Hoppe-Seyler 369:425-429, 1988, and Alexander et al., NUCLEIC ACIDS RES. 17:6739, 1989 (bovine β-lactoglobulin); and Vilotte et al., BIOCHIMIE 69:609-620, 1987 (bovine α-lactalbumin). The structure and function of the various milk protein genes are reviewed by Mercier & Vilotte, J. DAIRY SCI. 76:3079-3098, 1993.

Other promoters that are useful in the methods of the invention include inducible promoters. Generally, recombinant proteins are expressed in a constitutive manner in most eukaryotic expression systems. The addition of inducible promoters or enhancer elements provides temporal or spatial control over expression of the transgenic proteins of interest, and provides an alternative mechanism of expression. Inducible promoters include heat shock protein, metallothionien, and MMTV-LTR, while inducible enhancer elements include those for ecdysone, muristerone A, and tetracycline/doxycycline.

Eukaryotic Expression Vectors

Various mammalian cell culture systems can also be employed to express recombinant proteins. Examples of mammalian expression systems include selected mouse L cells, such as thymidine kinase-negative (TK) and adenine phosphoribosul transferase-negative (APRT) cells. Other examples include the COS-7 lines of monkey kidney fibroblasts, described by Gluzman, CELL 23:175 (1981), and other cell lines capable of expressing a compatible vector, for example, the C127, 3T3, CHO, HeLa and BHK cell lines. In particular, as regards yeasts, there may be mentioned yeasts of the genus Saccharomyces, Kluyveromyces, Pichia, Schwanniomyces, or Hansenula. Among the fungi capable of being used in the present invention, there may be mentioned more particularly Aspergillus ssp, or Trichoderma ssp.

Mammalian expression vectors will comprise an origin of replication, a suitable promoter and enhancer, and also any necessary ribosome binding sites, polyadenylation site, splice donor and acceptor sites, transcriptional termination sequences, and 5′ flanking non-transcribed sequences. DNA sequences derived from the SV40 viral genome, for example, SV40 origin, early promoter, enhancer, splice, and polyadenylation sites may be used to provide the required non-transcribed genetic elements.

Mammalian promoters include beta-casein, beta-lactoglobulin, whey acid promoter others include: HSV thymidine kinase, early and late SV40, LTRs from retrovirus, and mouse metallothionein-1. Exemplary mammalian vectors include pWLneo, pSV2cat, pOG44, pXT1, pSG (Stratagene) pSVK3, pBPV, pMSG, and pSVL (Pharmacia). In a preferred embodiment, the mammalian expression vector is pUCIG-MET. Selectable markers include CAT (chloramphenicol transferase).

The foregoing is not intended to have identified all of the aspects or embodiments of the invention nor in any way to limit the invention. The accompanying drawings, which are incorporated and constitute part of the specification, illustrate embodiments of the invention, and together with the description, serve to explain the principles of the invention. All publications and patent applications mentioned in this specification are herein incorporated by reference to the same extent as if each independent publication or patent application is specifically indicated to be incorporated by reference.

While the invention has been described in connection with specific embodiments thereof, it will be understood that it is capable of further modifications and this application is intended to cover any variations, uses, means or adaptations of the invention following the principles of the invention and including such departures from the present disclosure that come within known or customary practice within the art to which the invention pertains and may be applied to the essential features of the inventive concept.

In particular the methods of the invention provide a convenient, economic vehicle to produce antibodies, recombinant antibodies and other precious reagents for use in biosensor applications. The methods employing the device are extremely powerful in detecting pathologic organisms and screening for specific aerosolized chemical agents. Although the invention has been described with reference to the presently preferred embodiments, various modifications can be made without departing from the spirit of the invention. For example, the antibody array of the invention can also be used to screen samples from aqueous systems such as water supplies or in agricultural applications. Accordingly, the scope of the invention should be determined not by the embodiment(s) illustrated but by the following claims and their legal equivalents.

### LITERATURE CITED AND INCORPORATED BY REFERENCE

1. Abdel-Hamid, I et al., Highly Sensitive Flow-Injection Immunoassay System For Rapid Detection Of Bacteria, ANAL. CHIM. ACTA, 1999 399:99-108.
2. Alzari, P. M. et al., Three-Dimensional Structure of Antibodies. ANN. REV. IMMUNOL. 1988 6:555-80.
3. Anand, N. N. et al., Synthesis and Expression in Escherichia Coli of Cistronic DNA Encoding an Antibody Fragment Specific for a Salmonella Serotype B O-Antigen, GENE 1991 100:39-44.
4. Aubry, M. et al., (1972) Kinetics of the Inactivation of Human and Bovine Trypsins and Chymotrypsins by Alpha1-Antibodyase Inhibitor and of their Reactivation by Alpha-2-Macroglobulin, CLIN CHIM ACTA, 78: 371-380.
5. Baguisi A,(1999) et al., Production of Goats by Somatic Cell Nuclear Transfer, NATURE BIOTECH; 17: 456-461.
6. Baker W F Jr.,(1989) Clinical Aspects of Disseminated Intravascular Coagulation: A Clinician's Point Of View, SEMIN THROMB HEMOST. 15(1):1-57.
7. Barbera J. L. et al., Large-Scale Quarantine Following Bioterrorism in the United States, JAMA, Vol. 286, pp. 2711-17 (2001).
8. Burks, E. A. et al., Rapid, High-Yield Recovery of Recombinant Digoxin Binding Single Chain Fv from Escherichia Coli. BIOTECHNOL. PROG. 1995 11:112-14.
9. Campbell J, et al., Environmental Air Sampling To Detect Biological Warfare Agents, MIL MED 1999. 164:541-2.
10. Colcher D, et al., Effects of Genetic Engineering on the Pharmacokinetics of Antibodies. QJ NUCL MED 1999; 43:132-9.
11. Davis, G. T. et al., Single Chain Antibody (SCA) Encoding Genes: One-Step Construction and Expression in Eukaryotic Cells. BIO/TECHNOL. 1991 9:165-69.
12. Dubel S. et al., Bifunctional and multimeric Complexes of Streptavidin Fused to Single Chain Antibodies (scFv), J IMMUNOL METHODS 1995:178:201-9.
13. Ebersole, R. C. et al., Spontaneously Formed Functionally Active Avidin Monolayers on Metal Surfaces: A Strategy For Immobilizing Biological Reagents and Design of PiezoelectricBiosensors, J. AM. CHEM. SOC., 112; 3239-41 (1990).
14. Emanuel P. A., et al., Recombinant Antibodies: A New Reagent For Biological Agent Detection, BIOSENS BIOELECTRON, January 2000;14(10-11):751-9.
15. Falipou S. et al., New Use of Cyanosilane Coupling Agent for Direct Binding of Antibodies to Silica Supports: Physicochemical Characterization of Molecularly Bioengineered Layers, BIOCONJUG CHEM 1999, 10:346-53.
16. Haab B B, Methods and Applications of Antibody Microarrays in Cancer Research, PROTEOMICS 2003, 3:2116-22.
17. Harlow, E., and D. Lane, ANTIBODIES: A LABORATORY MANUAL. (Cold Spring Harbor, N.Y.: Cold Spring Harbor Laboratory. (1988)).
18. Holt, P. S., et al., Use of Negative Air Ionization For Reducing Airborne Levels of Salmonella Enterica Serovar Enteritidis in a Room Containing Infected Caged Layers. J. APPL. POULT. RES. 8:440-446. 1999.
19. Hruby, V. J. et al., Emerging Approaches in the Molecular Design of Receptor-Selective Peptide Ligands: Conformational, Topological and Dynamic Considerations, BIOCHEM. J. 1990, 268, 249-62.
20. Ivnitski, D et al., Biosensors for Detection of Pathogenic Bacteria, BIOSENS. BIOELECT., 1999 14: 599-624.
21. Jakobovits, A. L. et al., Expression of Human Immunoglobulin Loci-Derived YACs in Mice: Towards Mice Producing A Large Repertoire Of Human Antibodies. J. CELL. BIOCHEM. ABSTR. SUPPL. 18D 1994 (Abstract T-017):185.
22. Kabat, E. A., et al., in SEQUENCES OF PROTEINS OF IMMUNOLOGICAL INTEREST, (5th ed. Washington, D.C.: U.S. Public Health Service, National Institutes of Health (1991)).
23. Kang, A. S. et al., Antibody Redesign by Chain Shuffling From Random Combinatorial Libraries, PROC. NATL. ACAD. SCI. USA 1991 88:11120-23.
24. Kingsmore S. F. et al., Measuring Proteins on Microarrays. CURR OPIN BIOTECHNOL 2002, 13:14-19.
25. Lambert J. N. et al., The Synthesis of Cyclic Peptides. J. CHEM. SOC. PERKIN TRANS. 1 2001, 471-84.
26. Lantto J. et al., Chain Shuffling to Modify Properties of Recombinant Immunoglobulins. METHODS MOI BIOL 2002; 178:303-16.
27. Larrick, J. W. et al., Polymerase Chain Reaction Using Mixed Primers: Cloning Of Human Monoclonal Antibody Variable Region Genes From Single Hybridoma Cells. BIO/TECHNOL. 1989 7:934-938.
28. Loeffelholz M. J., et al., Comparison of PCR, culture, and direct fluorescent-antibody testing for detection of Bordetella Pertussis. JOURNAL OF CLINICAL MICROBIOLOGY. 37(9):2872-6. September 1999.
29. Maniatis, T. et al., MOLECULAR CLONING: A LABORATORY MANUAL. (2nd ed. Cold Spring Harbor, N.Y.: Cold Spring Harbor Laboratory (1989)).
30. McCafferty et al., Selection and Rapid Purification of Murine Antibody Fragments That Bind a Transition-State Analog by Phage Display, APPLIED BIOCHEM. & BIOTECH., 47:157-173 (1994).
31. Mitchell, B. W., et al., Reducing Airborne Pathogens, Dust, And Salmonella Transmission In Experimental Hatching Cabinets Using An Electrostatic Space Charging System. POULT. SCI. 81:49-55. 2002.
32. Nallur G. et al., Protein and Nucleic Acid Detection by Rolling Circle Amplification on Gel-Based Microarrays, J BIOMED MICRODEVICES 2003, 5:117-25.
33. Orlandi, R. et al., Cloning Immunoglobulin Variable Domains for Expression by the Polymerase Chain Reaction, PROC. NATL. ACAD. SCI. USA 86 (May 1989):3833-37.
34. Padlan, E. A., Anatomy of the Antibody Molecule, MOL. IMMUNOL. 31(3):169-217 (1994).
35. Perlee L. T. et al., Development and Standardization of Multiplexed Antibody Microarrays for Use in Quantitative Proteomics, PROTEOME SCIENCE 2004, 2:9.
36. Pluckthun A. et al., New Protein Engineering Approaches to Multivalent and Bispecific Antibody Fragments, IMMUNOTECHNOLOGY 1997; 3:83-105.
37. Reichmann, L. M. et al., Reshaping Human Antibodies for Therapy, NATURE (1988) 332:323-27.
38. Richardson, L. J., et al., Effect of an Electrostatic Space Charge System on Airborne Dust and Subsequent Potential Transmission Of Microorganisms to Broiler Breeder Pullets by Airborne Dust, AVIAN DIS. 47:128-133. 2003.
39. Robey, F. A., Bromoacetylated Synthetic Peptides: Starting Materials for Cyclic Peptides, Peptomers and Peptide Conjugates: In Methods in Molecular Biology. Vol 35, Peptide Synthesis Protocols. (Pennington, M. and Dunn, B., Eds., (Humana Press, Totowa, N.J.,)(1994)) pp 73-91.
40. Rowe C. A. et al., An Array Immunosensor for Simultaneous Detection of Clinical Analytes, ANAL CHEM 1999. 71:433-39.
41. Seo, K. H., et al., Bactericidal Effects of Negative Air Ions on Airborne and Surface Salmonella Enteritidis from an Artificially Generated Aerosol. J. FOOD PROT. 64:113-116.2001.
42. Shao W. et al., Optimization of Rolling Circle Amplified Protein Microarrays for Multiplexed Protein Profiling, J BIOMED BIOTECHNOL 2003, 5:299-307
43. Stenger D A, et al., Detection of Physiologically Active Compounds Using Cell-Based Sensors, TRENDS BIOTECHNOL 2001. 19:304-9.
44. Sundberg, S. A. et al., Spatially-Addressable Immobilization of Macromolecules on Solid Supports, J. AM. CHEM. SOC., 117, 12050-57 (1995).
45. Taki, S. et al., Targeted Insertion of a Variable Region Gene Into the Immunoglobulin Heavy Chain Locus, SCIENCE 1993 262:1268-70.
46. Wacker R. et al., Performance of Antibody Microarrays Fabricated by either DNA-Directed Immobilization, Direct Spotting, or Streptavidin-Biotin Attachment: A Comparative Study, ANAL BIOCHEM 2004, 330:281-87.
47. Whitlow M. B. et al., An Improved Linker For Single-Chain Fv With Reduced Aggregation and Enhanced Proteolytic Stability. PROTEIN ENG. (1993) 6(8):989-95.
48. Worn A., et al., Stability Engineering of Antibody Single-Chain Fv fragments. J MOL BIOL 2001; 305:989-1010.
49. Wu P. et al., Toward Immobilized Antibody Microarray Optimization: Print Buffer and Storage Condition Comparisons on Performance, BIOMED SCI INSTRUM 2004, 40:243-48.
50. Wu, X. C. et al., Efficient Production of a Functional Single-Chain Antidigoxin Antibody via an Engineered Bacillus Subtilis Expression-Secretion System, BIO/TECHNOL. 1993 11:71-76.

U.S. Patent Documents
Buechler, et al., U.S. Pat. No. 6,680,209
Chin, et al., U.S. Pat. No. 6,197,599
Fitzpatrick et al., U.S. Pat. No. 5,451,504
Kutzko et al. U.S. Pat. No. 6,268,487
Lennen et al., U.S. Pat. No. 5,567,627
Olson et al., U.S. Pat. No. 5,001,072

What is claimed is:

1. A method for detecting the presence of at least one biological agent of interest in a sample, said method comprising:
(a) combining said sample with a composition comprising a population of biosensor antibodies, wherein
i. said biosensor antibodies are bound to a solid support;
ii. the population of said biosensor antibodies comprises antibodies directed to at least one biological agent of interest in said sample;
(b) capturing any biological agents of interest;
(c) identifying any biological agents of interest present in said sample; and,
(d) reporting the capture of said at least one biological agent of interest.

2. The method according to claim 1, wherein said at least one biological agent of interest is present in the environment in an aerosolized form prior to capture in said sample.

3. The method according to claim 1, wherein said at least one biological agent of interest is present in the environment in an aqueous solution prior to capture in said sample.

4. The method according to claim 1, wherein said at least one biological agent of interest is present in the environment in an food substance prior to capture in said sample.

5. The method according to claim 1, wherein said at least one biological agent is contacted by biosensor calins instead of antibodies.

6. The method according to claim 1, wherein said at least one biological agent may contacted by a biosensor calins in addition to a biosensor antibody.

7. The method according to claim 1, wherein said population of biosensor antibodies represents at least two discrete subpopulations each directed towards the detection of a different biological agent of interest.

8. The method according to claim 1, wherein said biosensor antibodies are produced in the milk of transgenic mammals.

9. The method according to claim 1, wherein biosensor antibodies detect the presence of one or more pathogenic organisms.

10. The method according to claim 1, wherein said population of biosensor antibodies represents at least ten discrete subpopulations each directed towards the detection of a different biological agent.

11. The method according to claim 1, wherein said biological agents detected are polypeptides, proteins, chemical agents or DNA sequences produced by food contaminating organisms.

12. The method according to claim 1, further comprising:
immobilizing each of said populations of biosensor antibodies on said solid support at a known, predetermined position on said solid support such that each of said antibodies can be identified by the position where it is immobilized and upon capture of a target analyte can report such capture to a user; and,
a reporting means for the capture of a said target analyte;
wherein upon mobilization of each of said populations of biosensor antibodies on said solid support at a known, predetermined position said biosensor antibodies are also oriented so as to optimize capture of said target analyte.

13. The method of claim 1, wherein said reporting means utilizes a secondary antibody for detection and reporting.

14. The method of claim 1, wherein said reporting means comprises mass spectrometry.

15. The method of claim 1, wherein said reporting means comprises a visible indication.

16. The method of claim 1, wherein said reporting means comprises a qualitative indication.

17. The method of claim 1, wherein said reporting means comprises a quantitative indication.

18. The method of claim 1, wherein said reporting means comprises a fusion antibody with a detectable tag.

19. The method of claim 18, wherein said tag is a fluorescent antibody.

20. The method of claim 1, wherein said support is made of materials selected from the group consisting of nitrocellulose, nylon, polyvinylidene difluoride, mica, silicon, glass, metal, metal coating or plastics, and its derivatives.

21. The method of claim 1, wherein the number of said antibodies immobilized on said solid support ranges from 10 to 500 different kinds.

22. The method of claim 1, wherein the number of said antibodies immobilized on said solid support ranges from 500 to 1,000 different kinds.

23. The support of claim 20, further comprising patterning the surface with a film of hydrophobic molecules.

24. The support of claim 20 wherein said hydrophobic molecules include biotin or biotin derivatives to form a said uniform biological layer such that capture antibodies can be bound to the biotinylated surface.

25. The support of claim 20 wherein the metal or metal coating of said solid support is selected from the group consisting of platinum, silver, copper, gold and combinations thereof.

26. The support of claim 24 wherein the biotin-binding molecules are streptavidin or streptavidin derivatives attached to capture antibodies.

27. The method of claim 1 wherein said at least one biological agent of interest is a target biological analyte molecule selected from or produced by one the following group: pathogenic bacteria, colonic bacteria, viruses, parasites, bacterial toxins, fungi, enzymes.

28. The method of claim 1 wherein said at least one biological agent of interest includes molecular toxins or chemicals selected from the group including: ricin; sarin; soman, tabun; cyanogens; chloride and hydrogen chloride; oleoresin capsicum; arsene; chlorine, diphosgene; phosgene; distilled mustard, ethyldichloroarsine, mustard-lewisite mixture; nitrogen mustard; organophosphate pesticides; aflatoxin; Trichothecene mycotoxins, and the Staphylococcus enterotoxins A, B and C.

29. The method of claim 27 wherein the pathogenic organism of interest is a target biological agent of interest and is selected from group including: Bacillus Anthracis; Yersina Pestis; Yersina enterocolitica; Francisella Tularensis; Vibrio Cholerae; Vibrio parahemolyticus; Klebsiella species; Pseudomonas aeruginosa; Streptococci; Listeria; Cryptosporidium; Venezuelan Equine Encephalitis, Filoviridae (Ebola and Marburg viruses specifically); Brucella abortus; Brucella melitensis; Brucella suis; Nipah virus; Hendra virus (formerly called equine morbillivirus); Flaviviruses; Burkholderia mallei (formerly known as Pseudomonas mallei); Smallpox varieties or orthopoxvirus [two forms: variola major and variola minor]; Coxiella burnetii; Arenaviridae (Lassa fever, Argentine and Bolivian hemorrhagic fever), the Bunyaviridae (Hantavirus, Congo-Crimean hemorrhagic fever, Rift Valley fever, and Yellow fever) families; the Dengue hemorrhagic fever virus; Aeromonas sobria; Aeromonas hydrophila; Aeromonas caviae; Escherichia coli; Salmonella typhi; Salmonella paratyphi; Salmonella enteriditis; Salmonella cholera-suis; Salmonella typhimurium; Salmonella heidelberg; Shigella sonnei; Shigella flexneri; Shigella boydit; Shigella dysenteriae; Mycobacterium tuberculosis; Yersinia enterocolitca; Aeromonas hydrophila; Plesiomonas shigelloides; Campylobacteria jejuni; Campylobacreria coli; Bacteroides fragilis; Clostridia septicum; Clostridia perfingens; Clostridia botulinum; and Clostridia difficile.

30. The method of claim 1, wherein the number of biological agents of interest detected is at least 2.

31. The method of claim 1, wherein the number of biological agents of interest detected is in a range of between 10 and 100.

32. The method of claim 8, further comprising the production a single type of biosensor antibody in the milk of one or more transgenic mammals at a production volume of at least 3 grams per liter of milk produced.

33. The method of claim 8,,further comprising the production a single type of biosensor antibody in the milk of one or more transgenic mammals at a production volume of at least 10 grams per liter of milk produced.

34. The method of claim 12, wherein at least one subpopulation of said biosensor antibodies have a cyclized peptide added to their Fc sequence to provide for additional means of attachment and orientation to a solid support.

35. The method of claim 12, wherein at least one subpopulation said biosensor antibodies have at least one peptide added to their Fc sequence to provide for additional means of attachment and orientation to a solid support.

36. The method of claim 34 or 35, wherein said cyclized peptide or said peptide is between 5 and 15 amino acids in length.

37. The method of claim 12, wherein at least one subpopulation of said biosensor antibodies are truncated to provide for improved means of attachment and orientation to a solid support without substantially affecting their physiological ability to bind a target analyte.

38. The method of claim 8, further comprising the production a single type of biosensor antibody in the milk of one or more transgenic mammals wherein said biosensor antibody has a truncated sequence on its Fc end that will allow improved attachment to a solid support without substantially affecting their physiological ability to bind a target analyte.

39. The method of claim 1 wherein said at least one biological agent of interest is a target biological analyte molecule produced by a pathogenic organism found in food.

40. The method of claim 8, wherein the antibodies produced by a transgenic mammal have their amino acid sequences altered to provide for a cyclized peptide added to their Fc sequence to provide for additional means of attachment and orientation to a solid support.

41. The method of claim 8, wherein the antibodies produced by a transgenic mammal have their amino acid sequences altered to provide for at least one peptide added to their Fc sequence to provide for additional means of attachment and orientation to a solid support.

42. The method of claim 8, wherein the antibodies produced by a transgenic mammal have their amino acid sequences altered to provide for an antibody sequence that is truncated to provide for improved means of attachment and orientation to a solid support without substantially affecting its physiological ability to bind a target analyte.

43. A biosensor apparatus for detecting the presence of at least one biological agent of interest in a sample, said apparatus comprising:
(a) means for taking a sample and thereafter combining said sample with a composition comprising a population of biosensor antibodies, wherein
i. said biosensor antibodies are bound to a solid support;
ii. the population of said biosensor antibodies comprises antibodies directed to at least one biological agent of interest in said sample;
(b) means for capturing any biological agents of interest;
(c) means for identifying any biological agents of interest present in said sample; and,
(d) means for reporting the capture of said at least one biological agent of interest.

44. The apparatus according to claim 43, wherein said at least one biological agent of interest is present in the environment in an aerosolized form prior to capture in said sample.

45. The apparatus according to claim 43, wherein said at least one biological agent of interest is present in the environment in an aqueous solution prior to capture in said sample.

46. The apparatus according to claim 43, wherein said at least one biological agent of interest is present in the environment in a food substance prior to capture in said sample.

47. The apparatus according to claim 43, wherein said at least one biological agent is contacted by biosensor calins instead of antibodies.

48. The apparatus according to claim 43, wherein said at least one biological agent may contacted by a biosensor calins in addition to a biosensor antibody.

49. The apparatus according to claim 43, wherein said population of biosensor antibodies represents at least two discrete subpopulations each directed towards the detection of a different biological agent of interest.

50. The apparatus according to claim 43, wherein said biosensor antibodies are produced in the milk of transgenic mammals.

51. The apparatus according to claim 43, wherein biosensor antibodies detect the presence of one or more pathogenic organisms.

52. The apparatus according to ciaim 43, wherein said population of biosensor antibodies represents at least ten discrete subpopulations each directed towards the detection of a different biological agent.

53. The apparatus according to claim 43, wherein said biological agents detected are polypeptides, proteins, chemical agents or DNA sequences produced by food contaminating organisms.

54. The apparatus according to claim 43, further comprising:
immobilizing each of said populations of biosensor antibodies on said solid support at a known, predetermined position on said solid support such that each of said antibodies can be identified by the position where it is immobilized and upon capture of a target analyte can report such capture to a user; and,
a reporting means for the capture of a said target analyte;
wherein upon mobilization of each of said populations of biosensor antibodies on said solid support at a known, predetermined position said biosensor antibodies are also oriented so as to optimize capture of said target analyte.

55. The apparatus of claim 43, wherein said reporting means utilizes a secondary antibody for detection and reporting.

56. The apparatus of claim 43, wherein said reporting means comprises mass spectrometry.

57. The apparatus of claim 43, wherein said reporting means comprises a visible indication.

58. The apparatus of claim 43, wherein said reporting means comprises a qualitative indication.

59. The apparatus of claim 43, wherein said reporting means comprises a quantitative indication.

60. The apparatus of claim 43, wherein said reporting means comprises a fusion antibody with a detectable tag.

61. The apparatus of claim 60, wherein said tag is a fluorescent antibody.

62. The apparatus of claim 43, wherein said support is made of materials selected from the group consisting of nitrocellulose, nylon, polyvinylidene difluoride, mica, silicon, glass, metal, metal coating or plastics, and its derivatives.

63. The apparatus of claim 43, wherein the number of said antibodies immobilized on said solid support ranges from 10 to 500 different kinds.

64. The apparatus of claim 43, wherein the number of said antibodies immobilized on said solid support ranges from 500 to 1,000 different kinds.

65. The support of claim 62, further comprising patterning the surface with a film of hydrophobic molecules.

66. The support of claim 62 wherein said hydrophobic molecules include biotin or biotin derivatives to form a said uniform biological layer such that capture antibodies can be bound to the biotinylated surface.

67. The support of claim 62 wherein the metal or metal coating of said solid support is selected from the group consisting of platinum, silver, copper, gold and combinations thereof.

68. The support of claim 66 wherein the biotin-binding molecules are streptavidin or streptavidin derivatives attached to capture antibodies.

69. The apparatus of claim 43 wherein said at least one biological agent of interest is a target biological analyte molecule selected from or produced by one the following group: pathogenic bacteria, colonic bacteria, viruses, parasites, bacterial toxins, fungi, enzymes.

70. The apparatus of claim 43 wherein said at least one biological agent of interest includes molecular toxins or chemicals selected from the group including: ricin; sarin; soman, tabun; cyanogens; chloride and hydrogen chloride; oleoresin capsicum; arsene; chlorine, diphosgene; phosgene; distilled mustard, ethyldichloroarsine, mustard-lewisite mixture; nitrogen mustard; organophosphate pesticides; aflatoxin; Trichothecene mycotoxins, and the Staphylococcus enterotoxins A, B and C.

71. The apparatus of claim 69 wherein the pathogenic organism of interest is a target biological agent of interest and is selected from group including: Bacillus Anthracis; Yersina Pestis; Yersina enterocolitica; Francisella Tularensis; Vibrio Cholerae; Vibrio parahemolyticus; Klebsiella species; Pseudomonas aeruginosa; Streptococci; Listeria; Cryptosporidium; Venezuelan Equine Encephalitis, Filoviridae (Ebola and Marburg viruses specifically); Brucella abortus; Brucella melitensis; Brucella suis; Nipah virus; Hendra virus (formerly called equine morbillivirus); Flaviviruses; Burkholderia mallei (formerly known as Pseudomonas mallei); Smallpox varieties or orthopoxvirus [two forms: variola major and variola minor]; Coxiella burnetii; Arenaviridae (Lassa fever, Argentine and Bolivian hemorrhagic fever), the Bunyaviridae (Hantavirus, Congo-Crimean hemorrhagic fever, Rift Valley fever, and Yellow fever) families; the Dengue hemorrhagic fever virus; Aeromonas sobria; Aeromonas hydrophila; Aeromonas caviae; Escherichia coli; Salmonella typhi; Salmonella paratyphi; Salmonella enteriditis; Salmonella cholera-suis; Salmonella typhimurium; Salmonella heidelberg; Shigella sonnei; Shigella flexneri; Shigella boydit; Shigella dysenteriae; Mycobacterium tuberculosis; Yersinia enterocolitca; Aeromonas hydrophila; Plesiomonas shigelloides; Campylobacteria jejuni; Campylobacreria coli; Bacteroides fragilis; Clostridia septicum; Clostridia perfingens; Clostridia botulinum; and Clostridia difficile.

72. The apparatus of claim 43, wherein the number of biological agents of interest detected is at least 2.

73. The apparatus of claim 43, wherein the number of biological agents of interest detected is in a range of between 10 and 100.

75. The apparatus of claim 50, further comprising the production a single type of biosensor antibody in the milk of one or more transgenic mammals at a production volume of at least 3 grams per liter of milk produced.

76. The apparatus of claim 50, further comprising the production a single type of biosensor antibody in the milk of one or more transgenic mammals at a production volume of at least 10 grams per liter of milk produced.

77. The apparatus of claim 54, wherein at least one subpopulation of said biosensor antibodies have a cyclized peptide added to their Fc sequence to provide for additional means of attachment and orientation to a solid support.

78. The apparatus of claim 54, wherein at least one subpopulation said biosensor antibodies have at least one peptide added to their Fc sequence to provide for additional means of attachment and orientation to a solid support.

79. The apparatus of claim 76 or 77, wherein said cyclized peptide or said peptide is between 5 and 15 amino acids in length.

80. The apparatus of claim 54, wherein at least one subpopulation of said biosensor antibodies are truncated to provide for improved means of attachment and orientation to a solid support without substantially affecting their physiological ability to bind a target analyte.

81. The apparatus of claim 50, further comprising the production a single type of biosensor antibody in the milk of one or more transgenic mammals wherein said biosensor antibody has a truncated sequence on its Fc end that will allow improved attachment to a solid support without substantially affecting their physiological ability to bind a target analyte.

82. The apparatus of claim 43 wherein said at least one biological agent of interest is a target biological analyte molecule produced by a pathogenic organism found in food.

83. The apparatus of claim 50, wherein the antibodies produced by a transgenic mammal have their amino acid sequences altered to provide for a cyclized peptide added to their Fc sequence to provide for additional means of attachment and orientation to a solid support.

84. The apparatus of claim 50, wherein the antibodies produced by a transgenic mammal have their amino acid sequences altered to provide for at least one peptide added to their Fc sequence to provide for additional means of attachment and orientation to a solid support.

85. The apparatus of claim 50, wherein the antibodies produced by a transgenic mammal have their amino acid sequences altered to provide for an antibody sequence that is truncated to provide for improved means of attachment and orientation to a solid support without substantially affecting its physiological ability to bind a target analyte.

86. The apparatus of claim 43 further comprising an electrostatic impeller to aid in the collection of particulates for sampling.

87. The apparatus of claim 43 wherein the biosensor array is further comprised of replaceable units detachable from said apparatus each unit comprising known subpopulations of biosensor antibodies.

88. The apparatus of claim 43 further comprising where individual antibody subpopulations are immobilized at a known, predetermined position on said solid support such that one or more subpopulations can be removed from the biosensor antibody detection system and replaced individually.

89. The apparatus of claim 43 wherein said biosensor antibody signals are operatively connected to a reporting means and can report the detection of the target biological analyte upon antibody capture.

90. The method of claim 1 wherein said at least one biological agent of interest includes agents of interest including: vomiting agents, blister agents, blood agents, chemical warfare agents, choking agents, incapacitating agents and tear agents.

91. The apparatus of claim 43 wherein said at least one biological agent of interest includes agents of interest including: vomiting agents, blister agents, blood agents, chemical warfare agents, choking agents, incapacitating agents and tear agents.

92. A method for detecting the presence of at least one biological agent of interest in a sample, said method comprising:
(a) combining said sample with a composition comprising a population of biosensor calins, wherein
i. said biosensor calins are bound to a solid support;
ii. the population of said biosensor calins comprises calins directed to at least one biological agent of interest in said sample;
(c) identifying any biological agents of interest present in said sample; and,
(d) reporting the capture of said at least one biological agent of interest.

93. An apparatus for detecting the presence of at least one biological agent of interest in a sample, said method comprising:
(a) combining said sample with a composition comprising a population of biosensor calins, wherein
i. said biosensor calins are bound to a solid support;
ii. the population of said biosensor calins comprises calins directed to at least one biological agent of interest in said sample;
(b) capturing any biological agents of interest;
(c) identifying any biological agents of interest present in said sample; and,
(d) reporting the capture of said at least one biological agent of interest.
